(19) United States
(12) Patent Application Publication
(10) Pub. No.: US 2025/0099026 A1
(43) Pub. Date: Mar. 27, 2025

(54) Non-invasive Method and System for Assessing Joint Conditions

(71) Applicants: Georgia Tech Research Corporation, Atlanta, GA (US); Emory University, Atlanta, GA (US)

(72) Inventors: Omer Inan, Marietta, GA (US); Rahul Kant Goel, Atlanta, GA (US); Quentin Goossens, Atlanta, GA (US); Samer Mabrouk, Atlanta, GA (US); Goktug Ozmen, Atlanta, GA (US); Ajay Premkumar, Atlanta, GA (US)

(21) Appl. No.: 18/893,608

(22) Filed: Sep. 23, 2024

Related U.S. Application Data
(60) Provisional application No. 63/584,597, filed on Sep. 22, 2023.

(51) Int. Cl. A61B 5/00

(52) U.S. Cl. CPC A61B 5/4585; A61B 5/0051; A61B 5/7267

(57) ABSTRACT

An exemplary system and method that non-invasively assess the effusion state of a joint via the use of a non-invasive measurement device. The measurement device actively interrogates the bone connected to the joint and nearby tissue with mechanical or acoustic energy by applying an impulse or vibratory mechanical or acoustic energy into the bone to assess the response of the bone at its connecting joint and its proximate tissue. The measurement device may additionally interrogate the bone connected to the joint and nearby tissue with electrical energy by applying an impulse or vibratory electrical energy into the joint (e.g., knee) or its nearby tissue to assess the electrical response of the joint and its proximate tissue. In some embodiments, the sensors employ both mechanical interrogation and electrical interrogation of the joint for the non-invasive assessment, e.g., for bioimpedance characteristics of the tissue and joint.

### CROSS REFERENCE TO RELATED APPLICATIONS

This application claims priority to, and the benefit of, U.S. Provisional Patent Application No. 63/584,597, filed Sep. 22, 2023, entitled “Non-invasive Method and System for Assessing Joint Conditions,” which is hereby incorporated by reference herein in its entirety.

### BACKGROUND

Joint arthroplasty, also known as joint replacement surgery, is a surgical procedure that replaces a damaged joint with an artificial joint or prosthesis to restore joint function. Approximately 1-2% of all total joint arthroplasty (TJA) develop a prosthetic joint infection (PJI) following surgery. PJI can be challenging to diagnose following post-operation and often requires costly and resource-intensive surgical intervention for treatment.

Current diagnosis and subsequent treatment of PJ may employ a combination of clinical evaluations, similar to other types of injections, involving blood tests, for example, for peripheral non-specific biomarkers (erythrocyte sedimentation rate (ESR), c-reactive protein (CRP)) or the drawing (i.e., aspiration) of synovial fluid from the knee or local tissue for bacterial culture. Blood analysis may not detect infection until the infection is more widespread, and the aspiration of synovial fluid can itself lead to infection and other complications by introducing more trauma to the surgery site after surgery, which can delay the recovery of the patient.

Ultrasound imaging, computed tomography imaging, and magnetic resonance imaging are costly to perform and require a specialized technician.

There is a benefit to improving the assessment of joints, particularly of the knee, following surgery, and in other routine clinical assessments.

### SUMMARY

An exemplary system and method are disclosed that can non-invasively assess the effusion state of a joint via the use of a non-invasive measurement device. The exemplary system and method can non-invasively assess other joint states such as the development or progression of osteoarthritis, rheumatoid arthritis, meniscus/ligament damage, implant integrity, implant loosening, other infections or inflammation, and disease.

The exemplary system and method may be employed with machine learning or statistical analysis to provide an estimated effusion state, an elevated effusion state, or a joint condition. The measurement device may actively interrogate the bone connected to the joint and nearby tissue with mechanical or acoustic energy by applying an impulse or vibratory mechanical or acoustic energy into the bone to assess the response of the bone at its connecting joint and its proximate tissue. The measurement device may additionally interrogate the bone connected to the joint and nearby tissue with electrical energy by applying an impulse or vibratory electrical energy into the joint (e.g., knee) or its nearby tissue to assess the electrical response of the joint and its proximate tissue. In some embodiments, the sensors employ both mechanical interrogation and electrical interrogation of the joint for the non-invasive assessment, e.g., for bioimpedance characteristics of the tissue and joint. Other interrogation may be employed, e.g., light assessment, thermal assessment, and acoustics, all of which may be implemented in a portable device.

In some embodiments, the exemplary system and method can be employed to provide a reliable, non-invasive, inexpensive, objective, quick, and convenient assessment of joint pathologies and/or joint conditions. In some embodiments, the exemplary system and method can be employed to non-invasively pre-screen the joint (e.g., knee) for post-surgery complications, e.g., as reflected by the effusion state of the joint (e.g., knee) that can provide additional clinical input to a clinical to direct aspiration for synovial fluid, laboratory tests, or ultrasound. Post-operation assessments can, for example, be performed two weeks, six weeks, and three months following a joint arthroplasty surgery. The exemplary system can be employed, e.g., at the 2-week post-op visit, to assess for elevated or anomalous effusion or the development of effusion (e.g., joint space effusion) to direct further diagnosis, e.g., aspiration, for an infection. By exciting the structure of interest via mechanical or acoustic energy, various dynamic responses of the bone and proximate (nearby) tissue can assess the joint and tissue condition, including effusion (fluids in the knee or joint cavity).

The active assessment can be coupled with a passive assessment. In additional, the exemplary system and method may be used to non-invasively assess other joint conditions or joint prosthetic concerns, such as the development or progression of osteoarthritis, rheumatoid arthritis, meniscus/ligament damage, implant integrity, implant loosening, other infections or inflammation, and disease.

In some aspects, a system is disclosed, including an analysis system. The analysis system includes a processor; and a memory having instructions stored thereon, wherein execution of the instructions by the processor causes the processor to: receive measurement data acquired from a device configured to interrogate energy into a knee and proximate tissue of a joint arthroplasty patient; determine, by a trained AI model or statistical model, an estimated effusion state of the knee and proximate tissue; and output by a report or graphical user interface the estimated effusion state, wherein the effusion state is employed by a clinician to direct treatment or to diagnosis (e.g., aspiration) for infection (e.g., prosthetic joint infection) of the knee and proximate tissue.

In some aspects, the measurement data is acquired (e.g., via an accelerometer, vibration transducer, or force sensor) from impulse or vibratory mechanical energy applied to the knee and/or proximate tissue (e.g., to provide modal analysis of standing wave effects induced at the knee and proximate tissue by the applied impulse or vibratory mechanical energy, including boundary conditions of the bone).

In some aspects, the measurement data is acquired from an impulse or electrical energy applied (e.g., by pre-defined voltage or current) to the knee and/or proximate tissue.

In some aspects, the measurement data is acquired from a shaker, a bioimpedance sensor, or a combination thereof.

In some aspects, the trained AI model or statistical model includes a linear regression model.

In some aspects, the device is configured to induce swept-frequency cosine excitation mechanical input to the knee or proximate tissue, wherein the mechanical input has a primary frequency component between 200 Hz and 5000 Hz and is inducted for at least 20 seconds (e.g., 30 seconds or more).

In some aspects, the device was positioned at an anteromedial position on the knee, an anterior position of the knee, or at a tibial crest of the knee.

In some aspects, the analysis system is configured as a cloud infrastructure.

In some aspects, the analysis system is an edge device configured to operate with the device.

In some aspects, a method of non-evasively evaluating an effusion state of a knee or proximate tissue is disclosed. The method including; providing a measurement device positioned at an anteromedial position on the knee, an anterior position of the knee, or at a tibial crest of the knee, wherein the measurement device is configured to (i) direct an impulse or vibratory mechanical energy to the knee and/or proximate tissue and measure resulting impulse or vibratory mechanical energy, (ii) direct an electrical stimulus to the knee and/or proximate tissue and measure resulting electrical measurement for a measure of bioimpedance of the knee and/or proximate tissue, or (iii) a combination thereof; transmitting measurement data acquired from the measurement device to an analysis system configured to determine, by a trained AI model or statistical model, an estimated effusion state of the knee and proximate tissue; determining, by the trained AI model or statistical model, an estimated effusion state of the knee and proximate tissue; and outputting by a report or graphical user interface the estimated effusion state, wherein the effusion state is employed by a clinician to direct treatment or to diagnosis (e.g., aspiration) for an infection (e.g., prosthetic joint infection) of the knee and proximate tissue.

In some aspects, the method further includes: directing aspiration of the knee or proximate tissue when an estimated effusion volume is higher than a normal baseline effusion volume at a post-operation assessment of the knee or proximate tissue.

In some aspects, the measurement device is positioned when the knee is bent between 30°-60°.

In some aspects, the measurement data is acquired (e.g., via an accelerometer, vibration transducer, or force sensor) from impulse or vibratory mechanical energy applied to the knee and/or proximate tissue (e.g., to provide modal analysis of standing wave effects induced at the knee and proximate tissue by the applied impulse or vibratory mechanical energy, including boundary conditions of the bone).

In some aspects, the measurement data is acquired from an impulse or electrical energy applied (e.g., by pre-defined voltage or current) to the knee and/or proximate tissue.

In some aspects, the measurement data is acquired from a shaker, a bioimpedance sensor, or a combination thereof.

In some aspects, the trained AI model or statistical model includes a linear regression model.

In some aspects, the measurement device is configured to induce swept-frequency cosine excitation mechanical input to the knee or proximate tissue, wherein the mechanical input had a primary frequency component between 200 Hz and 5000 Hz and was inducted for at least 20 seconds (e.g., 30 seconds or more).

In some aspects, the analysis system is configured as a cloud infrastructure or an edge device configured to operate with the measurement device.

In some aspects, a system is disclosed. The system includes an analysis system including a processor; and a memory having instructions stored thereon, wherein execution of the instructions by the processor causes the processor to: receive measurement data acquired from a device configured to interrogate energy into a knee and proximate tissue of a joint arthroplasty patient; determine, by a trained AI model, an estimated joint condition of the knee and proximate tissue, wherein the estimated joint condition is selected from the group consisting of periprosthetic joint infections, implant integrity, implant loosening, and disease; and output by a report or graphical user interface the estimated joint condition, wherein the estimated joint condition is employed by a clinician to direct treatment or to diagnosis (e.g., for prosthetic joint infection, osteoarthritis, rheumatoid arthritis, meniscus/ligament damage, implant integrity, implant loosening, disease) of the knee and proximate tissue.

In some aspects, the device is configured with an accelerometer (e.g., wideband, low noise accelerometers), a force sensor, a thermal sensor, or combinations thereof.

### BRIEF DESCRIPTION OF DRAWINGS

FIGS. 1A-1C each shows an exemplary system to non-invasively assess the effusion state of a joint via the use of a non-invasive measurement device in accordance with an illustrative embodiment.

FIG. 1D shows an exemplary system to non-invasively assess the state of a joint via the use of a non-invasive measurement device in accordance with an illustrative embodiment.

FIG. 2 illustrates an exemplary method of non-invasively assessing the effusion state of a joint via the use of a non-invasive measurement device in accordance with an illustrative embodiment.

FIGS. 3A and 3B, respectively, show active mechanical sensing measurement, e.g., via active acoustics, and associated modeling, in accordance with an illustrative embodiment.

FIG. 4 shows active electrical sensing measurement, e.g., via active bioimpedance sensing, in accordance with an illustrative embodiment.

FIG. 5 shows an example method of non-invasive assessment for estimated effusion state or joint condition, e.g., as described in relation to FIGS. 1A-1D, in accordance with an illustrative embodiment.

FIGS. 6A-6H show details of example prototypes and results of a study conducted to develop and evaluate a non-invasive sensing system using active and passive joint acoustic emission as a biomarker of periprosthetic joint effusions.

FIGS. 7A-7B show details of example prototypes and results of a study conducted to develop and evaluate a non-invasive sensing system using active and passive joint acoustic emission as a biomarker of pre-periprosthetic joint effusions.

FIGS. 8A-8E show details of example prototypes and results of a study conducted to develop and evaluate high input-impedance buffer for dry-electrode bioimpedance analysis.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [41] refers to the 41st reference in the list. All references cited and discussed in this specification are incorporated herein by reference in their entirety and to the same extent as if each reference was individually incorporated by reference.

### Example System

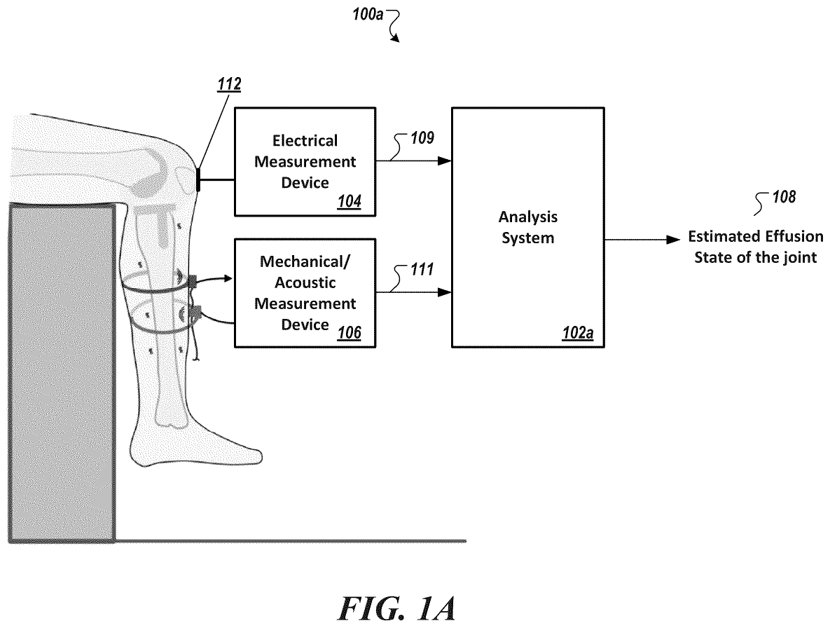

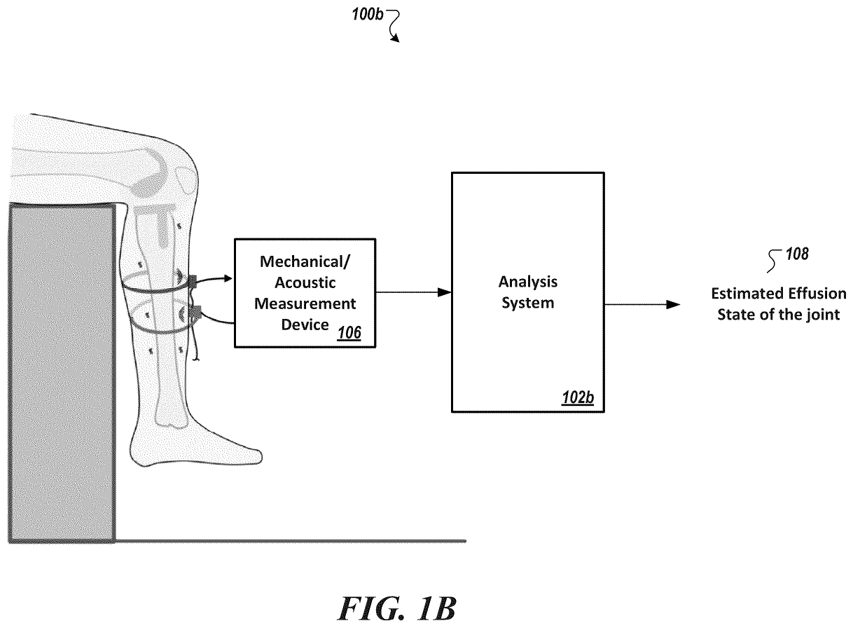

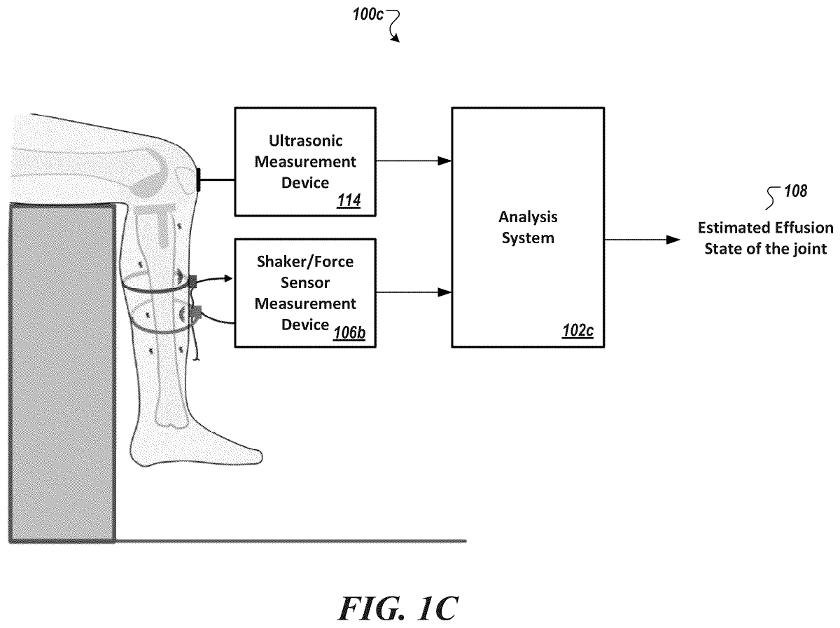

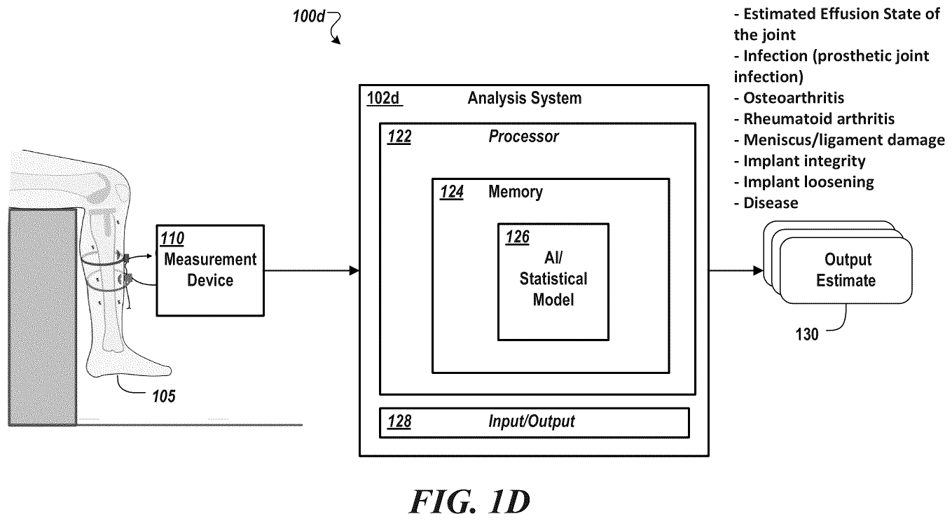

FIGS. 1A-1C each shows an exemplary system 100 (shown as 100a, 100b, 100c, 100d) configured to non-invasively assess the effusion state of a joint via the use of non-invasive sensors in accordance with an illustrative embodiment.

Example #1. In the example shown in FIG. 1A, the system 100a includes an analysis system 102 (shown as 102a) configured to receive measurement data from a non-invasive electrical measurement device 104 and a non-invasive mechanical measurement device 106 (shown as “Mechanical/Acoustic Measurement Device” 106) to determine an estimated effusion state 108 of the joint or proximal tissue.

The analysis system 102a includes a processor; and a memory having instructions stored thereon, wherein execution of the instructions by the processor causes the processor to receive measurement data 109, 111 acquired from one or more devices (e.g., 104, 106) configured to interrogate energy into a knee and proximate tissue of a joint arthroplasty patient; determine, by a trained AI model or statistical model, an estimated effusion state of the knee and proximate tissue; and output, via a report or graphical user interface, the estimated effusion state, wherein the effusion state is employed by a clinician to direct treatment or to diagnosis (e.g., aspiration) for infection (e.g., prosthetic joint infection), or sign thereof, of the knee and proximate tissue. The analysis system 102a may be configured as a cloud infrastructure that communicates directly, or through an edge device, over a network with the measurement device 104, 106 to receive the measurement data. In other embodiments, the analysis system 102 is configured as the edge device configured to operate with the measurement devices 104, 106.

Mechanical/Acoustic Measurement Device. In FIG. 1A, the measurement data 111 is acquired, e.g., via an accelerometer, vibration transducer, or force sensor, from impulse or vibratory mechanical energy applied to the knee and/or proximate tissue by the mechanical measurement device 106, e.g., that can be used to perform by the analysis system 102 a modal analysis of standing wave effects induced at the knee and proximate tissue by the applied impulse or vibratory mechanical energy, including boundary conditions of the bone.

In some embodiments, the mechanical measurement device 106 is positioned on a bone region in connection with the joint, or to which the joint is located at its boundary and is configured to introduce/generate a swept-frequency cosine mechanical excitation to the knee or proximate tissue by way of bone conduction excitation. The mechanical excitation may have multiple frequency components, with at least one or two of the primary frequency components being between 100 Hz and 5000 Hz and induced for up to a minute, e.g., at least 20 seconds e.g., 30 seconds or more. In some embodiments, the mechanical excitation is around: 100 Hz, 200 Hz, 300 Hz, 400 Hz, 500 Hz, 600 Hz, 700 Hz, 800 Hz, 900 Hz, 1000 Hz, 1100 Hz, 1200 Hz, 1300 Hz, 1400 Hz, 1500 Hz, 1600 Hz, 1700 Hz, 1800 Hz, 1900 Hz, 2000 Hz, 2100 Hz, 2200 Hz, 2300 Hz, 2400 Hz, 2500 Hz, 2600 Hz, 2700 Hz, 2800 Hz, 2900 Hz, 3000 Hz, 3100 Hz, 3200 Hz, 3300 Hz, 3400 Hz, 3500 Hz, 3600 Hz, 3700 Hz, 3800 Hz, 3900 Hz, 4000 Hz, 4100 Hz, 4200 Hz, 4300 Hz, 4400 Hz, 4500 Hz, 4600 Hz, 4700 Hz, 4800 Hz, 4900 Hz, or 5000 Hz. In some embodiments, the mechanical excitation is less than 200 Hz, for example, between 1 Hz and 5000 Hz, for example, for at least 30 seconds, at least 40 seconds, at least 50 seconds, at least 60 seconds, or more.

For the knee joint in a knee joint assessment, the mechanical measurement device 106 may be positioned at an anteromedial position on the knee, an anterior position of the knee, or at a tibial crest of the knee. Other-like positions can be used at other joints in which impulse or vibration energy can be introduced into a bone and, by way of bone conduction excitation, induce standing wave effects on the bone to which the joint of the bone provides a boundary for a modal analysis at the joint.

Electrical Measurement Device. The measurement data 109 is acquired from an impulse or electrical energy applied, e.g., by pre-defined voltage or current, e.g., to provide bioimpedance measurement to the knee and/or proximate tissue. The measurement system 104 may employ dry electrodes (e.g., silver silicone electrodes) or gel-electrodes that are placed on the surface of the knee or its proximal tissue. A current or voltage stimulus may be actively introduced by measurement system 104 via a first set of electrodes to which a corresponding voltage or current may be measured via a second set of electrodes or via the same set of electrodes. The measurement system 104 may include a high input-impedance buffer that can mitigate common-mode and/or input-impedance mismatch errors.

For the knee joint in a knee joint assessment, the electrical measurement device 104 may have electrodes 112 positioned at the knee.

The electrical measurement device 104 may introduce a voltage between 100 mV and 6000 mV and a frequency between 1 kHz and 10 kHz. In some embodiments, the applied voltage to the electrodes has an average voltage of about 100 mV_{AC}, 100 mV_{AC}, 200 mV_{AC}, 300 mV_{AC}, 400 mV_{AC}, 500 mV_{AC}, and 600 mV_{AC}. In some embodiments, the applied voltage has multiple frequency components with at least one or two of the primary frequency component being between 1 kHz and 100 kHz, e.g., around 1 kHz, 2 kHz, 3 kHz, 4 kHz, 5 kHz, 6 kHz, 7 kHz, 8 kHz, 9 kHz, 10 kHz, 11 kHz, 12 kHz, 13 kHz, 14 kHz, 15 kHz, 16 kHz, 17 kHz, 18 kHz, 19 kHz, 20 kHz, 21 kHz, 22 kHz, 23 kHz, 24 kHz, 25 kHz, 26 kHz, 27 kHz, 28 kHz, 29 kHz, 30 kHz, 31 kHz, 32 kHz, 33 kHz, 34 kHz, 35 kHz, 36 kHz, 37 kHz, 38 kHz, 39 kHz, 40 kHz, 41 kHz, 42 kHz, 43 kHz, 44 kHz, 45 kHz, 46 kHz, 47 kHz, 48 kHz, 49 kHz, 50 kHz, 51 kHz, 52 kHz, 53 kHz, 54 kHz, 55 kHz, 56 kHz, 57 kHz, 58 kHz, 59 kHz, 60 kHz, 61 kHz, 62 kHz, 63 kHz, 64 kHz, 65 kHz, 66 kHz, 67 kHz, 68 kHz, 69 kHz, 70 kHz, 71 kHz, 72 kHz, 73 kHz, 74 kHz, 75 kHz, 76 kHz, 77 kHz, 78 kHz, 79 kHz, 80 kHz, 81 kHz, 82 kHz, 83 kHz, 84 kHz, 85 kHz, 86 kHz, 87 kHz, 88 kHz, 89 kHz, 90 kHz, 91 kHz, 92 kHz, 93 kHz, 94 kHz, 95 kHz, 96 kHz, 97 kHz, 98 kHz, 99 kHz, or 100 kHz.

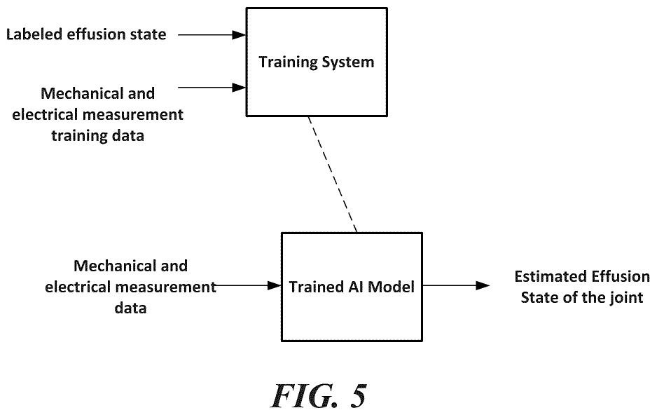

Trained AI or statistical model. The measurement data 109, 111 are employed by the trained AI model or statistical model, e.g., comprising linear regression model or transfer function or equation derived therefrom. Other ML models may be employed that are trained using labeled data comprising effusion state and the input data comprising the mechanical measurement or the electrical measurement, e.g., as described in relation to FIG. 5.

Post-surgery knee assessment. Referring still to FIG. 1A, system 100a can be used to non-invasively pre-screen the joint (e.g., knee) for post-surgery complications, e.g., as reflected by the effusion state of the joint (e.g., knee) that can provide additional clinical input to a clinical to direct aspiration for synovial fluid, laboratory tests, or ultrasound. Post-operation assessments can, for example, be performed two weeks, six weeks, and three months following a joint arthroplasty surgery. The exemplary system can be employed, e.g., at the 2-week post-op visit, to assess for elevated or anomalous effusion or the development of effusion (e.g., joint space effusion) to direct further diagnosis, e.g., aspiration, for an infection. By exciting the structure of interest via mechanical or acoustic energy, various dynamic responses of the bone and proximate (nearby) tissue can assess the joint and tissue condition, including effusion (fluids in the knee or joint cavity).

In some embodiments, the measurement data acquired from other measurement devices may be employed, for example, vibration analysis and acoustic emission-associated measurement equipment, joint acoustic emission measurement equipment, or other types of mechanical analysis referenced herein.

Example #2. In the example shown in FIG. 1B, system 100b includes an analysis system 102 (shown as 102b) configured to receive measurement data from the non-invasive mechanical measurement device 106 to determine an estimated effusion state 108 of the joint or proximal tissue. The measurement device 106 may be the same as that described in relation to FIG. 1A.

The analysis system 102b may also include a processor and a memory having instructions stored thereon, wherein execution of the instructions by the processor causes the processor to receive measurement data 111 acquired from one or more devices (e.g., 106) configured to interrogate energy into a knee and proximate tissue of a joint arthroplasty patient; determine, by a trained AI model or statistical model, an estimated effusion state of the knee and proximate tissue; and output, via a report or graphical user interface, the estimated effusion state, wherein the effusion state is employed by a clinician to direct treatment or to diagnosis (e.g., aspiration) for infection (e.g., prosthetic joint infection), or signs thereof, of the knee and proximate tissue. The analysis system 102b may be configured as a cloud infrastructure or an edge device configured to operate with the measurement device 106.

In FIG. 1B, the measurement data 111 may be acquired, e.g., via an accelerometer, vibration transducer, or force sensor, from impulse or vibratory mechanical energy applied to the knee and/or proximate tissue by the mechanical measurement device 106, e.g., that can be used to perform by the analysis system 102b a modal analysis of standing wave effects induced at the knee and proximate tissue by the applied impulse or vibratory mechanical energy, including boundary conditions of the bone.

Example #3. In the example shown in FIG. 1C, the system 100c includes an analysis system 102 (shown as 102c) configured to receive measurement data from a non-invasive measurement 114 employing ultrasonic measurements (shown as “Ultrasonic Measurement Device” 114) and a non-invasive mechanical measurement device 106 (shown as “Shaker/Force Sensor Measurement Device” 106b) to determine an estimated effusion state 108 of the joint or proximal tissue.

The ultrasound measurements device (referred to as device 114a) employs an ultrasonic sensor configured to introduce acoustic energy having a frequency over 20 kHz into the knee or proximate tissue. An example of the device includes a piezoelectric device or ultrasonic transducer.

Example #4. In the example shown in FIG. 1D, the system 100d includes a measurement device 110 that measures and provides measurement data related to a patient's joint condition to an analysis system 102 (shown as 102d). The computing device outputs an estimation of the joint condition.

In some embodiments, the analysis system 102d includes a processor 122 and a memory 124 having instructions stored thereon, wherein execution of the instructions by the processor 122 causes the processor to receive the measurement data acquired from the measurement device 110 configured to interrogate energy into a knee and proximate tissue of a joint arthroplasty patient; determine, by a trained AI model or statistical model, an estimated effusion state of the knee and proximate tissue; and output by a report or graphical user interface the estimated effusion state, wherein the effusion state is employed by a clinician to direct treatment or to diagnosis (e.g., aspiration) for infection (e.g., prosthetic joint infection) of the knee and proximate tissue.

In some embodiments, the measurement device 110 includes an accelerometer, vibration transducer, or force sensor. In some embodiments, the measurement device 110 includes a shaker, a bioimpedance sensor, or a combination thereof. In some embodiments, the measurement device 110 is configured to induce swept-frequency cosine excitation mechanical input to the knee or proximate tissue, wherein the mechanical input has a primary frequency component between 200 Hz and 5000 Hz and is inducted for at least 20 seconds or other ranges described herein, e.g., described in relation to FIG. 1A. For example, the mechanical input may be applied for at least 30 seconds, at least 40 seconds, at least 50 seconds, at least 60 seconds, or more. In some embodiments, the measurement device is positioned at an anteromedial position on the knee, an anterior position of the knee, and/or at a tibial crest of the knee.

In some embodiments, the measurement data is acquired from impulse or vibratory mechanical energy applied to the knee and/or proximate tissue by the measurement device 110. In some embodiments, the measurement data is used to provide modal analysis of standing wave effects induced at the knee and proximate tissue by the applied impulse or vibratory mechanical energy, including boundary conditions of the bone. In some embodiments, the trained AI model or statistical model comprises a linear regression model for modal analysis.

In some embodiments, the modal analysis includes one or more frequency response functions. In some embodiments, an input feature of the trained linear regression model is band power. In some embodiments, the trained linear regression model predicts an effusion volume. In some embodiments, the modal analysis is carried out for modes within a frequency range below 10 kHz, e.g., below 5 kHz.

In some embodiments, the analysis system 102d is configured as a cloud infrastructure. In some embodiments, the analysis system is an edge device configured to operate with the measurement device 110.

In some embodiments, the analysis system 102d includes a processor 122 and a memory 124 having instructions stored thereon, wherein execution of the instructions by a processor causes the processor to receive measurement data acquired from the measurement device 110 configured to interrogate energy into a knee and proximate tissue of a patient; determine, by a trained AI or statistical model 126, an estimated joint condition of the knee and proximate tissue, wherein the estimated joint condition is selected from the group consisting of periprosthetic joint infections, implant integrity, implant loosening, and disease; and output by a report or graphical user interface the estimated joint condition, wherein the estimated joint condition is employed by a clinician to direct treatment or to diagnosis (e.g., for prosthetic joint infection, osteoarthritis, rheumatoid arthritis, meniscus/ligament damage, implant integrity, implant loosening, disease) of the knee and proximate tissue.

In some embodiments, the measurement device 110 is configured with an accelerometer, a force sensor, a thermal sensor, or combinations thereof. For example, the measurement device 110 is configured with a wideband, low-noise accelerometer.

### Example Method

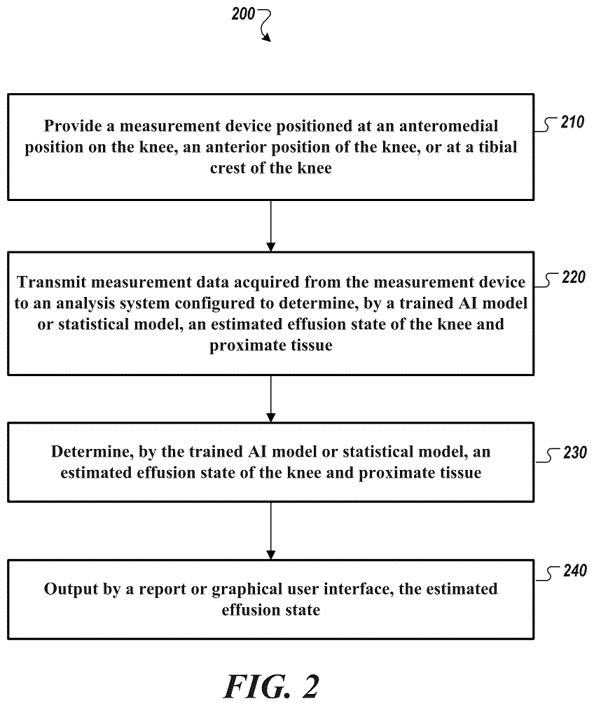

FIG. 2 illustrates an exemplary method of non-invasively assessing the effusion state of a joint via the use of a non-invasive measurement device in accordance with an illustrative embodiment. In other embodiments, the method may be similarly applied to the non-invasive assessment of other conditions, e.g., joint pathologies (e.g., osteoarthritis, rheumatoid arthritis, meniscus/ligament damage) and/or joint conditions (e.g., following joint replacement surgeries (e.g., periprosthetic joint infections, implant integrity, implant loosening, particle disease), among others described or referenced herein.

Referring now to FIG. 2, an exemplary method 200 of non-evasively evaluating the effusion state of a knee or proximate tissue is shown. In some embodiments, the method 200 includes providing a measurement device positioned at an anteromedial position on the knee, an anterior position of the knee, or at a tibial crest of the knee 210, wherein the measurement device is configured to (i) direct an impulse or vibratory mechanical energy to the knee and/or proximate tissue and measure resulting impulse or vibratory mechanical energy, (ii) direct an electrical stimulus to the knee and/or proximate tissue and measure resulting electrical measurement for a measure of bioimpedance of the knee and/or proximate tissue, or (iii) a combination thereof; transmitting measurement data acquired from the measurement device to an analysis system configured to determine, by a trained AI model or statistical model, an estimated effusion state of the knee and proximate tissue 220; determining, by the trained AI model or statistical model, an estimated effusion state of the knee and proximate tissue 230; and outputting by a report or graphical user interface the estimated effusion state 240, wherein the effusion state is employed by a clinician to direct treatment or to diagnosis (e.g., aspiration) for an infection (e.g., prosthetic joint infection) of the knee and proximate tissue.

In some embodiments, for active vibrational sensing, the effusion volume V can be determined per Equation 1.

$$V = A * BP + C \qquad ( Eq . l )$$

In Equation 1, V is the effusion volume (ml), and BP is the normalized band power feature (−). Example values for the coefficients A and C for the linear regression model are A=20 and C=37 based on certain experiments. Band power may be determined as the area under a FRF curve within a specified frequency range. The normalization may be done by subtracting the baseline BP. Examples of FRF curve calculations are provided later herein.

For passive sensing, the effusion volume V can be determined per Equation 2.

$$V = A * {T}_{1} + B * {T}_{2} + C \qquad ( Eq . 2 )$$

In Equation 2, V=effusion volume (ml), T₁ is the first normalized temporal moment (T) (−), and T₂ is the second normalized temporal moment (Kₜ) (−). Example values for the coefficients A, B, and C (ml) for the linear regression model are A=10, B=13, and C=38.

In some embodiments, the method further includes directing aspiration of the knee or proximate tissue when an estimated effusion volume is higher than a normal baseline effusion volume at a post-operation assessment of the knee or proximate tissue.

In some embodiments, the measurement device is positioned when the knee is bent between 30°-60°. For example, the knee is bent at 30°, 35°, 40°, 45°, 50°, 55°, or 60°.

In some embodiments, a system to monitor or assess joint pathologies and/or joint conditions includes a first energy source configured to transmit energy proximate a joint (e.g., via a miniature shaker or manual stress application); two or more sensors configured to measure a transmitted energy proximate the joint for assessment of joint pathologies (e.g., osteoarthritis, rheumatoid arthritis, meniscus/ligament damage) and/or joint conditions (e.g., following joint replacement surgeries (e.g., periprosthetic joint infections, implant integrity, implant loosening, particle disease)), wherein a first sensor includes a force sensor, and wherein the force sensor is configured to measure an input energy from the first energy source, and wherein a second sensor is configured to measure an output acceleration of the transmitted energy; and a computing device including a processor and a memory, the memory including instructions that, when executed by the processor, cause the processor to provide an assessment of joint pathologies and/or joint conditions from the two or more sensors.

In some embodiments, the assessment includes a modal analysis of the transmitted energy forming a standing wave. In some embodiments, the assessment includes a trained AI model or statistical model comprising a linear regression model for modal analysis.

In some embodiments, the modal analysis includes one or more frequency response functions. In some embodiments, an input feature of the trained linear regression model is band power. In some embodiments, the trained linear regression model predicts an effusion volume. In some embodiments, the modal analysis is carried out for modes within a frequency range below 10 kHz, e.g., below 5 kHz.

In some embodiments, the assessment of joint pathologies and/or joint conditions includes an indication of effusion. For example, the indication of effusion progression is an effusion type, an effusion volume, or both.

In some embodiments, the transmitted energy is electrical energy, mechanical energy, or a combination thereof. In some embodiments, the first energy source includes a mechanical shaker or manual stress application. In some embodiments, the first energy source provides a vibrational energy. In some embodiments, the vibrational energy includes a frequency band of 200 Hz to 5000 Hz. In some embodiments, the vibrational energy is provided periodically.

In some embodiments, the system further includes a second energy source, wherein the second energy source is a current or voltage source of a bioimpedance device. In some embodiments, a third and a fourth sensor are configured to measure the output acceleration of the transmitted energy. In some embodiments, the first energy source and the two or more sensors are enclosed in housing configured to allow for dynamic coupling.

In some embodiments, the two or more sensors are wearable sensors.

In some embodiments, a system for detecting and/or monitoring joint effusion is used at weekly or monthly post-operative clinician visits of a patient. For example, the system is used for detecting and/or monitoring knee joint effusion weekly and/or monthly following a total or partial knee replacement. The clinician may utilize the non-invasive system to detect whether fluid buildup is developing around the post-operative knee. In some examples, the system is used after an indication of possible infection from a blood test, wherein the non-invasive system is used to detect fluid buildup in the post-operative knee. Further, if effusion is detected, the clinician may order a follow-up aspiration of the fluid for further testing for infection. In other embodiments, the non-invasive system is used to detect effusion in the post-operative knee and distinguish between septic and a-septic fluid.

In some embodiments, measurements are taken at several time points post-operation, for example, at two- or four-weeks post-operation or one-, two- three-, or four-months post-operation.

### Example Mechanical Assessment of the Knee Joint

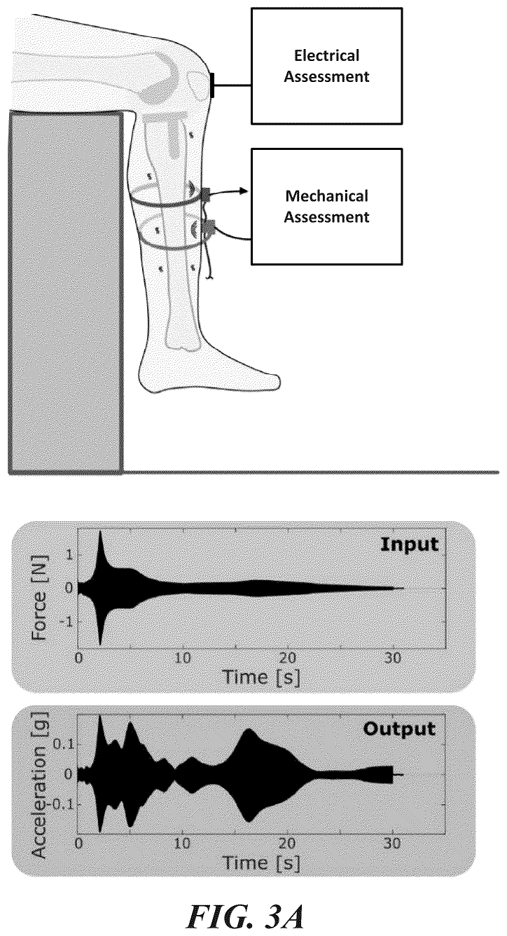

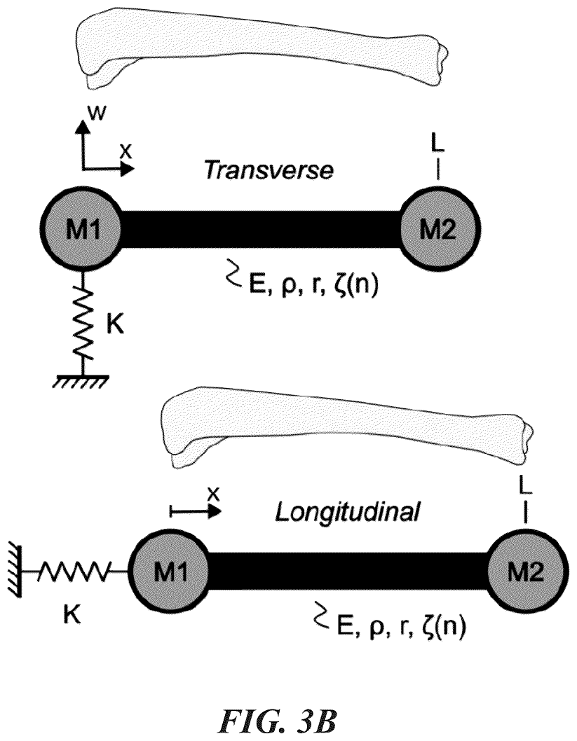

FIG. 3A shows active mechanical sensing measurement, e.g., via active acoustics. FIG. 3B illustrates a transverse and longitudinal analytical model of the knee-tibia-foot system.

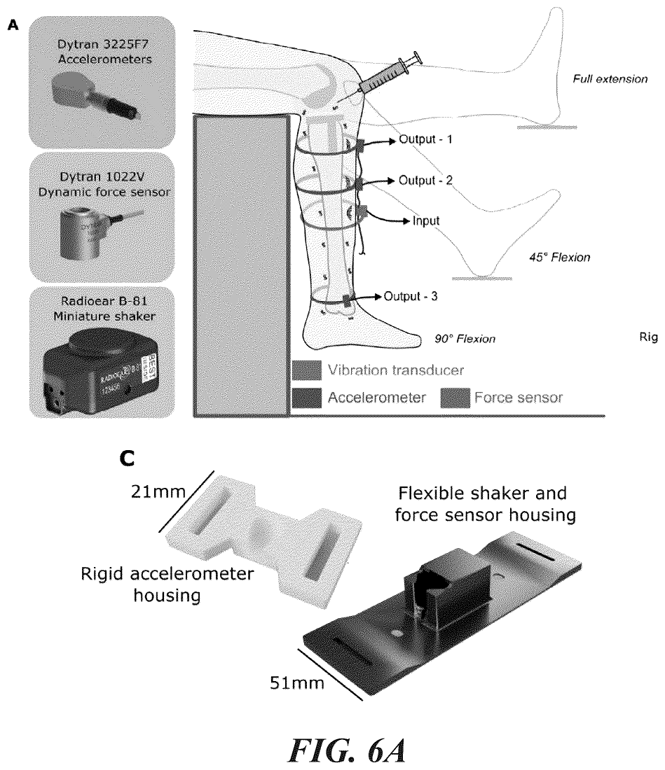

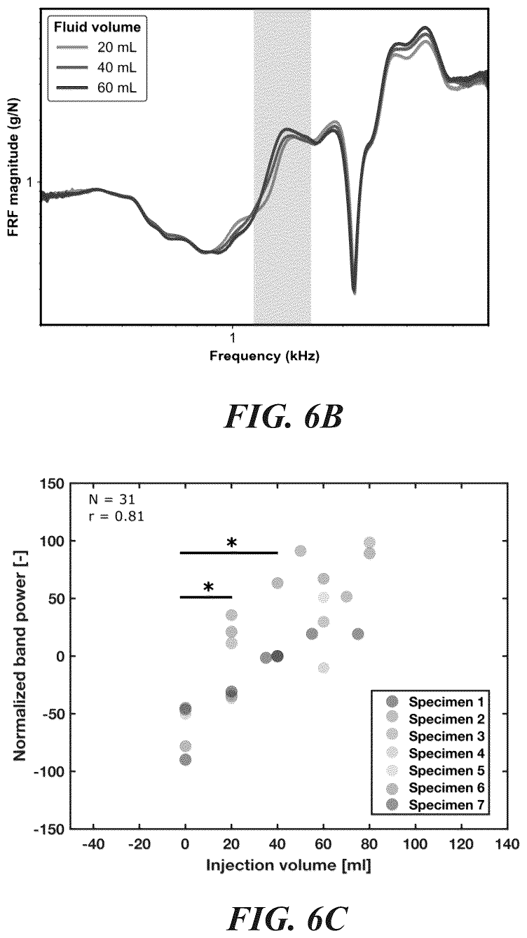

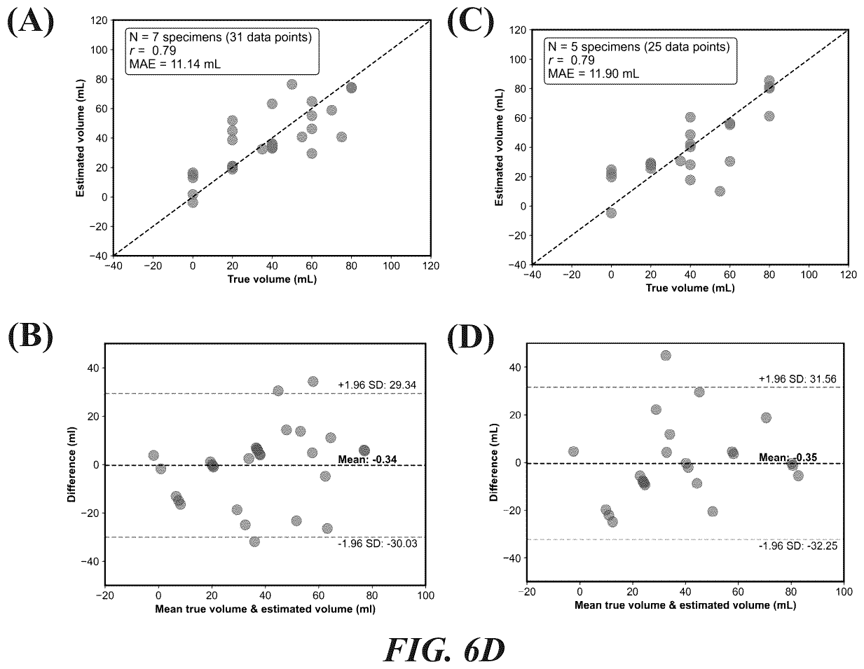

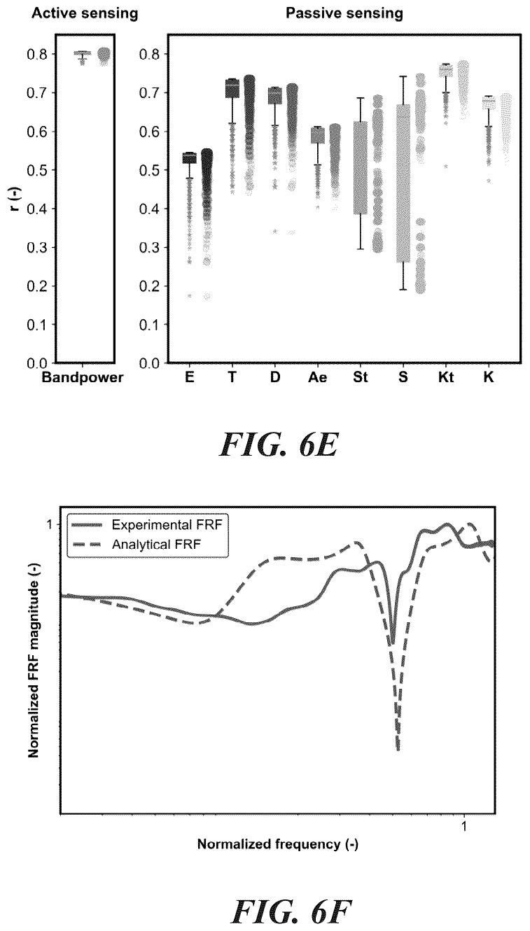

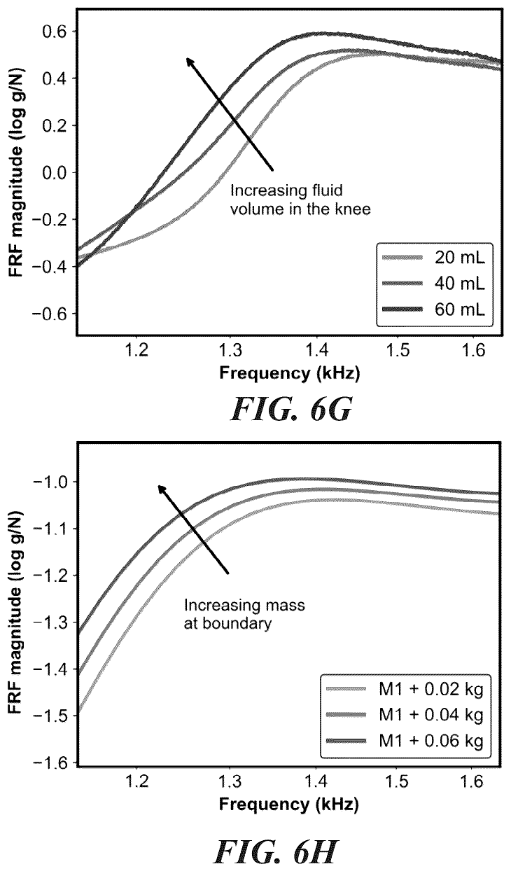

In FIG. 3A, active mechanical sensing measurement, e.g., via active acoustics, is introduced, e.g., per the method described in relation to FIG. 2. In an example, the anteromedial position on the knee, an anterior position of the knee, or at a tibial crest of the knee may be excited by a miniature vibration motor attached to the skin. At the location, the thin, soft tissue layer minimally impedes the transmission of vibrations from the motor. A linear swept-frequency cosine excitation signal with a fixed 3.3V amplitude with a frequency band between 200 Hz and 5000 Hz may be applied, e.g., for a total duration of 30 seconds or other excitation time described herein. Two miniature dynamic force sensors, or other sensors described herein, may be positioned, including a first sensor positioned between the vibration motor and the skin to measure the input force of the vibration motor and a second sensor positioned proximal to the knee. The mechanical sensing measurement may additionally or alternatively include an acceleration positioned proximal to the knee or other positioned described herein, e.g., described in relation to FIG. 6A. The accelerometer may have a broad bandwidth (2 Hz-5 kHz or 10 kHz), high sensitivity (e.g., 100 mV/g), low noise floor (e.g., 700 g rms).

Analysis of the mechanical measurement may include an input-output frequency response functions (FRFs) assessment. Power and cross-spectral densities may be determined, e.g., in the excitation frequency band, 200 Hz-5000 Hz with a 1 Hz resolution.

To estimate the fluid volume in the joint space, a linear regression model may be used. Other analysis or ML learning operations, as described herein, may be used.

Analytical beam model. The knee-tibia-foot system may be modeled as an Euler-Bernoulli beam with specific boundary conditions. FIG. 3B shows the knee modeled as a mass and a spring at the left boundary, while the foot is modeled as a mass at the right end. For the active sensing, the transverse vibrations of the beam may be solved with the orientations chosen to align with the input force direction that generated the analyzed vibrations (active sensing: motor excitation perpendicular to the tibia, passive sensing: the impulses generated longitudinally on the tibial tray during the varus-valgus stress applications). The fluid volume in the knee joint (effusion) may be assessed as an increase in the mass of M1 in correspondence with the mass of the fluid. The equation of motion of the presented Euler-Bernoulli beam is provided per Equation 3.

$$EI \frac{{\partial }^{4} w ( x , t )}{\partial {x}^{4}} + ρ A \frac{\partial w ( x , t )}{\partial t} = f ( x , t ) \qquad ( Eq . 3 )$$

In Equation 3, E is Young's modulus, I is the moment of area of the cross-section, p the mass density and A is the cross-sectional area of the beam. In the above equation, w(x,t) is the transverse displacement of the beam; f(x,t) is the transverse force applied on the beam. The transverse displacement of the beam w(x,t) may be decomposed using the separation of variables per Equation 4.

$$w ( x , t ) = {\sum }_{n} {W}_{n} ( x ) {T}_{n} ( t ) \qquad ( Eq . 4 )$$

In Equation 4, Wₙ(x) is the shape function, and Tₙ(t) is the time-dependent function for the n^{th} mode. Modal damping, ((n), per mode may be introduced to the FRF solution.

Active acoustic differs from passive acoustics in the utilization of an input source to actively excite the structure of interest. In contrast, passive acoustics focuses on the intrinsic vibrations produced by the joint, such as its natural movements. The movements may be inherently dependent on the patient, leading to uncontrolled variations between different individuals. Active acoustics have benefits over passive acoustics in allowing for the repeated excitation of the structure with a controlled and sufficient amount of energy (high signal-to-noise ratio) that does not rely on any movements of the patient. The controlled excitation of active acoustic can also permit the concentration on specific dynamic ranges, e.g., to provide a comprehensive approach that facilitates more sensitive detection of minor structural alterations in the joint compared to the currently available techniques.

An exemplary system and method are disclosed for monitoring a patient's joint condition using sensors positioned near the joint of interest that can capture active acoustics and electrical bioimpedance from the joint and surrounding tissues.

An example implementation includes monitoring periprosthetic effusions following joint replacement surgery. The exemplary system and method can provide an assessment of musculoskeletal injuries, facilitate early intervention, and mitigate the progression of joint pathologies.

The active acoustics (AA) analysis is a vibration-based technique that analyzes the forced vibrational response of a structure to monitor its mechanical characteristics using at least one input vibration source (motor), including a sensor to measure the input (e.g., force, acceleration), and at least one output sensor (motion transducer) that captures the dynamic response because of the excitation. The input and output signals are used to calculate frequency response functions (FRFs) that reflect the dynamic characteristics of the joint. AA can be used to assess joint characteristics such as stiffness, mass, and damping.

The kth-order temporal moments M^{k} about a reference time tₛ may be determined [11] per Equation 5.

$${M}_{k} ( {t}_{s} ) = {\int }_{- ∞}^{+ ∞} {( t - {t}_{s} )}^{k} {y ( t )}^{2} d t , k = 0 , 1 , 2 , \qquad ( Eq . 5 )$$

### Example Bioimpedance Electrical Assessment

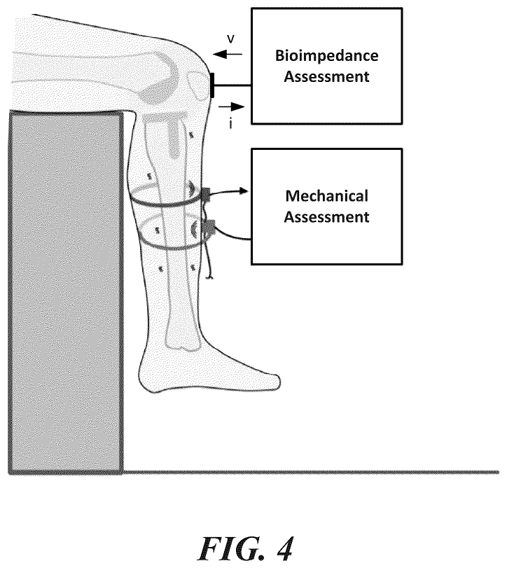

FIG. 4 shows active electrical sensing measurement, e.g., via active bioimpedance sensing. Active bioimpedance sensing may be introduced, e.g., per the method described in relation to FIG. 2. The electrical bioimpedance (EBI) sensor can measure the electrical conductivity of the tissue. Additional sensors that can capture physiological changes (e.g., local tissue temperature) can also be included in the system.

The EBI sensor signals (and acoustic mechanical signals) can be collected during either a static joint position or during joint movement.

Bioimpedance sensing has sensitivities to alterations in fluid volume within the knee joint. The presence of conductive fluid augments the current's path, thereby diminishing the overall impedance of the measured tissue. The variable conductivity of the fluid may also exert an influence on changes in bioimpedance. Notably, bioimpedance may respond distinctively to identical volumes of two distinct fluids possessing different conductivities, e.g., to distinguish between infection and normal synovial fluid or normal postoperative swelling. The supplementary volume assessment, via active sensing, can be used to enhance bioimpedance's capacity to differentiate between these fluid types by leveraging their discrepant conductivity profiles.

The bioimpedance modality may be used to optimize the parameters for the acoustic mechanical modality. For example, bioimpedance may be used to grossly locate the fluid, and then the active vibration-sensing transducers can be more optimally placed on the joint to maximize the accuracy of the measurement. Conversely, active vibration sensing can provide some indication of the location of fluid within the joint space, and the bioimpedance electrodes can be placed accordingly at the optimal locations. The parameters of the measurements can also be used to optimize the other measurement modality. For example, if active vibration sensing shows that there is a high likelihood of extracellular fluid buildup, then the bioimpedance frequencies can be selected to be lower rather than higher.

Adding to the above, dynamic bioimpedance recordings can provide insight into whether the fluid is located intraarticular or extraarticular (i.e., inside the joint space or outside). For example, the bioimpedance waveforms recorded during flexion-extension are expected to differ depending on the fluid's location. Analyzing them can help identify freely moving fluid or edema, as opposed to a fluid that is trapped inside the joint space, informing the active vibration experimental setup.

If both intraarticular and extraarticular fluid are present, the combined information can also help distinguish between the two. For instance, active vibration sensing can be normalized using the bioimpedance data, allowing for an accurate estimation of the fluid volume of interest, whether it is intraarticular or extraarticular.

### Example Machine Learning Analysis

FIG. 5 shows an example method of non-invasive assessment for estimated effusion state or joint condition, e.g., as described in relation to FIGS. 1A-1D.

In an example, active vibration and bioimpedance are combined as features in machine learning (ML) algorithms. Because active vibration sensing and bioimpedance are both independently informative about fluid volume, location, and properties, merging them within a regression or classifier ML model can yield highly accurate predictions. Potential outcomes of the ML model can include fluid volume, fluid location, and fluid type. The algorithms can include classical ML techniques, where manual feature engineering is used, or deep learning techniques, where the signals are inputted directly to neural networks or other architectures that can directly learn the information from the measured data.

Machine Learning. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model a pattern in the data. In a semi-supervised model, the model learns a function that maps an input (also known as feature or features) to an output (also known as a target) during training with both labeled and unlabeled data.

Neural Networks. An artificial neural network (ANN) is a computing system including a plurality of interconnected neurons (e.g., also referred to as “nodes”). The instant disclosure contemplates that the nodes can be implemented using a computing device (e.g., a processing unit and memory as described herein). The nodes can be arranged in a plurality of layers such as an input layer, an output layer, and optionally one or more hidden layers. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. The instant disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. The instant disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. The instant disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is a supervised classification model that classifies new data points based on similarity measures (e.g., distance functions). The k-NN classifiers are trained with a data set (also referred to herein as a “dataset”) to maximize or minimize a measure of the k-NN classifier's performance during training. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

Referring to FIG. 5, a training system 502 is configured using mechanical and electrical measurement training data labeled with effusion state data. The effusion data may be acquired via controlled insertion/injection of fluid in a joint body structure, e.g., of a cadaver or a phantom. In some embodiments, simulated effusion data are generated via generative adversarial network or physics-based simulation to generate simulated data that may be employed for the training.

### Experimental Result and Additional Examples

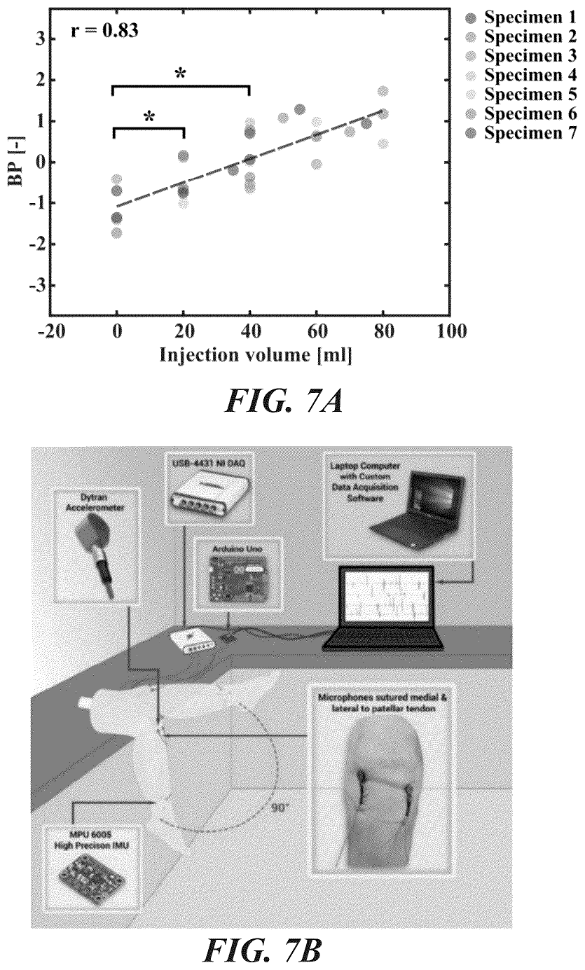

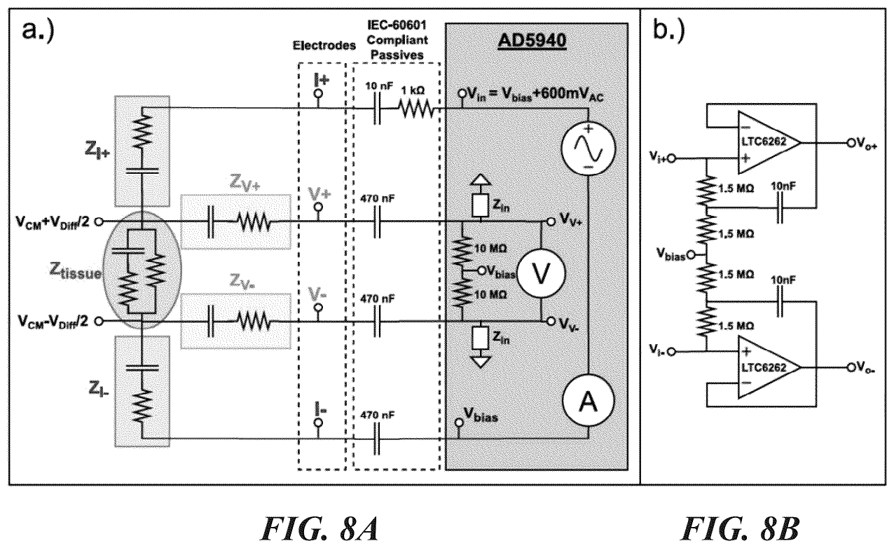

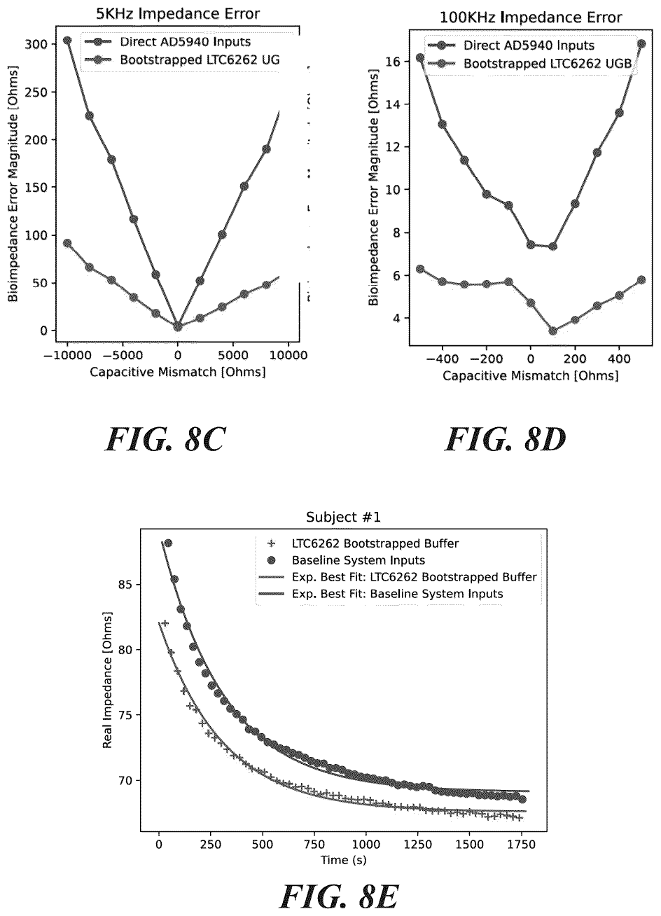

A study was conducted to develop a non-invasive sensing system using active and passive joint acoustic emission as a biomarker of periprosthetic joint effusions (Example #A). Details and results are provided in relation to FIGS. 6A-6H. The study also evaluated the early periprosthetic joint effusions (Example #B). Details and results are provided in relation to FIGS. 7A-7B. The study also developed and evaluated a high input-impedance buffer for dry-electrode bioimpedance analysis (Example #C). Details and results are provided in relation to FIGS. 8A-8E.

### Example #A: Non-Invasive Sensing of Active and Passive Joint Acoustic Emissions as a Biomarker of Periprosthetic Joint Effusions

A study was conducted to develop a non-invasive sensing system using active and passive joint acoustic emission as a biomarker of periprosthetic joint effusions (Example #A). Details and results are provided in relation to FIGS. 6A-6H. FIG. 6A illustrates the custom-designed housings for the transducers to ensure proper dynamic coupling with the cadaver specimen.

FIG. 6B illustrates examples of FRFs measured from a specimen resulting from the output measurement at the mid-diaphysis in 45° flexion over three different effusion stages. The most sensitive frequency band (1390.5±249.5 Hz) over all specimens is highlighted.

FIG. 6C illustrates a scatter plot between the normalized band power and the injected volume in the joint space using the frequency range (1390.5±249.5 Hz) that resulted in the highest correlation. * p<0.05.

FIG. 6D, subpanels A and B, shows the results of the LOSO-CV for the active sensing, scatter plot between the true volume and estimated volume (subpanel A), and Bland-Altman plot showing the mean error and the 95% limit of agreement (subpanel B). The trained linear regression model estimated the injected volume in the joint space with a Pearson's r of 0.79, an MAE of 11.1 mL, and a maximum absolute error of 34.3 mL.

FIG. 6D, subpanels C and D, shows the results of the LOSO-CV for the passive sensing, scatter plot between the true volume and estimated volume (subpanel C), and Bland-Altman plot showing the mean error and the 95% limit of agreement (subpanel D). The trained linear regression model estimated the injected volume in the joint space with a Pearson's r of 0.79, an MAE of 11.9 mL, and a maximum absolute error of 44.9 mL.

FIG. 6E shows the boxplot and corresponding scatter representation of the feature robustness analysis for both the active sensing and passive sensing features. Gaussian noise (mean=0, SD=2.5% of the total signal length) was added to the time segment, which led to the maximum correlation with the effusion volume for each feature. Pearson's r distribution represents the robustness of each feature to the selected time segment.

FIG. 6F illustrates a comparison of the experimentally measured (20 mL, as shown in FIG. 6B) and analytically simulated FRFs. The frequency axis is normalized to the first 5 modes.

FIG. 6G shows a detailed view of an example of the experimentally measured FRFs for three injected volumes (same example as FIG. 6B). FIG. 6H shows an analytically simulated FRFs when adding two increments of 0.02 kg to M1, corresponding to the injected volumes of the experiment. Both FRFs are shown in the most sensitive frequency band (1390.5±249.5 Hz).

The study developed and evaluated two non-invasive vibration-based approaches to monitor periprosthetic joint effusions: active and passive sensing. The active sensing technology utilized a miniature external vibration source to excite the tibia in a controlled manner. The dynamic force and the resulting output acceleration were captured using skin-mounted transducers to calculate input-output FRFs. Passive sensing was used manually performed varus-valgus stress applications to generate transient vibrations, ‘clicks’, in the artificial knee joint that are captured using skin-mounted accelerometers. Joint space effusions were simulated by injecting saline and bacteria solutions in the joint space with 20 mL increments. All transducers used in the study were attached to the skin and thus non-invasive. Features that were sensitive to the fluid in the joint space were extracted from both the active and passive sensing signals, spectral band power, and temporal features, respectively.

The active sensing technology proved to be highly sensitive to the fluid in the joint space. The band power in the 1390.5±249.5 Hz band correlated well (0.81) with the fluid volume. The study then used the feature in combination with the specimen's BMIs to train a linear regression model as a predictor of the fluid volume in the joint space. The model performed well with an MAE of 10.9 mL between the predicted and true fluid volume in the joint space. Other models were contemplated, though not yet implemented.

The results of the study show the use of active and passive vibrational techniques to monitor periprosthetic joint effusions. It is contemplated that such non-invasive methods may be incorporated into a sleeve or brace, could improve the timely detection of acute PJI, reducing the morbidity of delayed PJI treatment, and help confirm PJI eradication, and thus guide patient-specific treatment protocols.

Total Joint Arthroplasty discussion. Total joint arthroplasty (TJA) is a highly effective treatment for end-stage hip and knee joint disease, improving patients' quality of life. However, 1-2% of TJA patients develop prosthetic joint infections (PJI), posing significant health and economic challenges. PJI diagnosis is complex, often relying on clinical signs, nonspecific biomarkers, and synovial fluid cultures. The study explored non-invasive active vibration analysis and passive acoustic emission analysis for PJI monitoring.

The instant study was a controlled ex vivo study having seven cadaveric specimens with knee replacements that simulated small periprosthetic joint effusions by injecting saline and bacteria solutions into the joint space. The study employed active sensing to noninvasively stimulate the tibia with a miniature shaker, while passive sensing induced vibrations by manual stress applications, as examples of the exemplary system and method. The resulting vibrations were captured using wideband, low-noise miniature accelerometers. Spectral features from the frequency response functions were extracted from the active recordings, and temporal features were analyzed from the passive recordings.

Both methods were shown to be sensitive to the fluid in the joint space. Linear regression models were developed using the most informative features to estimate the joint fluid volume. When incorporating the most sensitive active sensing features and specimen BMI, the model achieved a Pearson's r of 0.81 and a mean absolute error of 10.9 mL. The results of the study demonstrated the utility of vibration-based techniques to monitor periprosthetic joint effusions. It is contemplated that these non-invasive techniques can lead to wearable devices for joint health monitoring, enabling PJI detection and personalized treatment plans, potentially improving patient outcomes, and reducing PJI-related healthcare costs.

Vibration-based techniques may offer a solution to the current status quo regarding early detection and monitoring of the development of PJI. The study investigated the use of both vibration analysis using an excitation source (active sensing) and AE-based techniques (passive sensing) to monitor the early development of joint space effusions related to PJI in the knee joint. Both active and passive techniques were compared in the study.

A cadaveric study was conducted to assess the utility of active and passive acoustic sensing to monitor small effusions in the joint space of the knee. The experimental work was supplemented by an analytical beam model to obtain a qualitative understanding of the effect of knee effusions on the dynamic response of the knee-tibia system. The model provided a means to qualitatively validate the experimental findings and offers the possibility to evaluate the effect of different mechanical changes on the observed vibrational response of the knee-tibia system.

It is contemplated that the vibration-based techniques may be used in a low-cost, non-invasive, and effective solution to conduct surveillance for PJI by providing a piece of objective point-of-care information on the presence and characteristics of a knee effusion or potentially identify the presence and characteristics of biofilm on the surface of an implant.

### Methods

Specimens and effusion protocol. Seven fresh-frozen hip-to-toe cadaver specimens were included in the study. Fresh-frozen specimens are commonly used in orthopedic training and ensure representative mechanical properties for an in vivo model. The specimens were obtained from individuals with an average age of 80.6±10.2 years and a BMI of 26.0±4.9 kg/m². The specimens were fully thawed overnight prior to the experiments. All seven specimens had knee replacements: three cruciate retaining TKAs, three posterior stabilizing TKAs, and one unicondylar partial knee arthroplasty. Hand-guided flexion-extension cycles were performed prior to testing to ensure proper joint flexibility and that the specimens were fully thawed. The specimens were positioned on a surgical table and secured to the table at the thigh using fabric bands. Different levels and types of effusion stages were simulated by injecting up to 80 mL with 20 mL increments of saline and methicillin-susceptible Staphylococcus aureus (MSSA, 108 CFU/mL) solutions into the joint space. The fluid was injected in the joint space through the anteromedial and anterolateral portal of the knee using a syringe with an 18-gauge needle. The skin temperature of the specimen was recorded during each measurement to ensure that temperature did not influence the recordings.

Active sensing protocol. Active vibrations were recorded at each effusion stage while the specimens were in an unsupported 900 and supported 450 flexion and full extension position. The tibia was excited by a miniature vibration motor (B-81, Radioear, Denmark) attached to the skin at the medial surface of the mid-diaphysis. At the location, the thin, soft tissue layer minimally impedes the transmission of vibrations from the motor to the tibia. A linear swept-frequency cosine excitation signal with a fixed 3.3 V amplitude, a frequency band between 200 Hz and 5000 Hz, and a total duration of 30 seconds was used to feed the shaker to excite the tibia. A miniature dynamic force sensor (1022V, Dytran, USA) was placed between the vibration motor and the skin to measure the input force of the vibration motor. The force sensor has excellent dynamic properties (50 kHz resonant frequency), high sensitivity (22.5 mV/N), a low noise floor (0.018 N rms), and a low noise floor (0.018 N rms).

The output acceleration was measured perpendicular to the skin using lightweight uniaxial accelerometers (3225F7, Dytran, USA) at three locations along the tibia: the tibial tuberosity, the medial surface of the mid-diaphysis approximately 2.5 cm proximal to the input, and the medial malleolus. These accelerometers have a broad bandwidth (2 Hz to 5 kHz or 10 kHz), high sensitivity (100 mV/g), low noise floor (700 g rms), miniature size, and low weight (1 gram). Both the sensors and the motor were attached to the skin using double-sided adhesive tape (Elizabeth Craft Designs, Inc., CO) in combination with a custom-designed housing with an elastic band surrounding the leg to ensure a proper and consistent attachment to the specimen. The elastic bands provided the backing force to the transducers, which has been shown to improve the dynamic coupling [38].

The accelerometer housings were 3D-printed out of a rigid material (polylactic acid, PLA) and included a layer of backing foam to decouple the resulting vibrations from the housing while keeping the sensor in place. Depending on the measurement location, different angles (0° and 35°) for the elastic band loops of the housing were utilized to ensure proper fit with the anatomical shape of the leg. The shaker/force sensor housing was 3D-printed out of a flexible material (thermoplastic polyurethane (TPU), NinjaFlex, Manheim, PA) to ensure a good fit at the mid-diaphysis. The housings that were used originated from a pilot study that involved various housing designs [39]. The pilot study showed that the selected housings resulted in a proper dynamic coupling between the input and output transducers and the tibia. The accelerometers, force sensor, and shaker were connected to a multi-channel input/output data acquisition system (USB 4431, National Instruments, Austin, TX). A customized Matlab (Mathworks, Natick, MA) script was used to acquire the data and feed the input signal to the shaker. All the recorded data were sampled at 50 kHz. FIG. 6A shows an overview of the experimental setup.

Signal processing and feature extraction. Input-output frequency response functions (FRFs) were calculated from the recorded input force and output acceleration for each output measurement location. The Hi FRF estimator method was chosen as a means to reduce the influence of random noise on the output measurement due to averaging the cross-spectral density [11]. The power and cross-spectral densities to calculate the FRFs were estimated using Welch's method in the excitation frequency band; 200 Hz-5000 Hz with a 1 Hz resolution [40]. Next, the power in various bands was extracted from each recorded FRF's to investigate their sensitivity to the simulated joint effusion. The band power is determined by computing the area under the FRF curve within the specified frequency band. To investigate the sensitivity of the feature to the fluid in the joint space, various frequency bands were considered to calculate band power. The lower and upper limits of the band of interest were varied over the total frequency range (200 Hz-5000 Hz). The extracted absolute band powers for each specimen were then normalized by subtracting the absolute band power measured at 40 mL for the corresponding specimen. This allowed the specimen-specific differences (offsets) of the absolute band power to be reduced. The 40 mL value was chosen as a baseline because it was the lowest common effusion stage that was present over all specimens.

The normalized band power values obtained at each effusion stage were then correlated across all specimens with the corresponding fluid injected into the joint space. The correlation helped identify the optimal frequency band, output measurement location, and leg position that led to the strongest relationship with the fluid in the joint space (i.e., the highest correlation coefficient). A Pearson's correlation was used for the analysis. Within-specimen correlation coefficients were also calculated and reported. A Wilcoxon rank sum test was used to compare the band power values at low effusion levels (20 mL and 40 mL) with the baseline (0 mL).

Next, to estimate the fluid volume in the joint space, a linear regression model was trained and validated using the previously selected most sensitive band power and demographic features (age and BMI) of the specimen. These demographic features were included to compensate for possible differences in mechanical properties between the specimens due to age or BMI. A leave-one-specimen-out cross-validation (LOSO-CV) approach was used to test the generalizability of the linear regression model. Pearson's correlation coefficients and the mean and maximum absolute error of the model were calculated and reported.

Analytical beam model. Numerical methods such as finite element (FE) analysis are widely used in engineering applications to better understand the vibrational or modal behavior of mechanical structures. These numerical models, also called digital twins, offer a solution to extensive experimental testing that is expensive and time consuming. Complex FE models of the tibia have been built and validated to study its vibrational behavior [21], [41]. While numerical models offer the possibility to solve complex structural models, they require a precise determination of the geometry and material properties to provide meaningful quantitative results. Alternatively, these complex mechanical structures can be approximated by a simplified model that can be solved in an analytical framework. While such a model is unable to precisely replicate the mechanical behavior of complex structures, it can provide valuable qualitative insight into and understanding the vibrational behavior of complex structures. For the study, the study chose to simulate the knee-tibia-foot system as an Euler-Bernoulli beam with specific boundary conditions (FIG. 3B). The knee is modeled as a mass and a spring at the left boundary, while the foot is modeled as a mass at the right end. For the active sensing, the study solved the transverse vibrations of the beam, while for the passive sensing, the study solved the longitudinal vibrations of the beam. These orientations were chosen to align with the input force direction that generated the analyzed vibrations (active sensing: motor excitation perpendicular to the tibia, passive sensing: the impulses generated longitudinally on the tibial tray during the varus-valgus stress applications). The fluid volume in the knee joint is simulated by increasing the mass of M1 in correspondence with the mass of the fluid. The equation of motion of the presented Euler-Bernoulli beam per Equations 4 and 5.

Modal damping, ζ(n), per mode, is introduced to the FRF solution. An overview of the model's properties values, which are based on average values found in literature, is shown in Table 1.

TABLE 1
Value of the properties of the Analytical Model

| Property | Value | |
|---|---|---|
| E | 10 | GPa |
| ρ | 2850 | kg/m3 |
| ζ(n) | [20, 20, 10, 10, 10, 10] % | |
| r | 0.017 | m |
| L | 0.365 | m |
| M1 | 0.07 | kg |
| M2 | 0.6 | kg |
| K | 1 | N/m |

### Results

Active sensing. A total of 35 effusion stages over all specimens were included in the study. Four data points were excluded due to a combination of insufficient measurement quality and being identified as statistical outliers. The temperature of all specimens remained constant during the entire experimental protocol. The normalized band power, derived from the input-output FRF for the different frequency ranges, was compared to the fluid introduced into the joint space for each leg position. The maximum Pearson's r between the normalized band power and the fluid in the joint space was found to be 0.81. The maximum correlation was observed when considering the frequency range of 1390.5±249.5 Hz, using the FRF generated from the output measurement at the mid-diaphysis of the tibia (output 2 in FIG. 8B), while the leg was positioned at a 45° flexion angle. FIG. 6B shows the FRFs of a representative specimen to illustrate of how the FRFs are affected by the fluid in the joint space.

FIG. 6C shows the corresponding scatter plot between the normalized band power and the fluid in the joint space across all specimens. The within-specimen Pearson's correlation coefficients (r) varied between 0.73 and 1.00. The band power corresponding to 20 mL and 40 mL injected volume showed to be significantly higher than the baseline (0 mL) (p<0.05). When comparing pure saline and pure bacteria solution at the 20 mL effusion stage, no significant difference was found between the two.

Regression Model. The normalized band power resulting from the 1390.5±249.5 Hz band was then selected as a feature to estimate the fluid in the joint space using a linear regression model. LOSO-CV was used to train and test the linear regression model. The model predicted the true fluid volume with a Pearson's r of 0.79 and an MAE of 11.1 mL and a maximum absolute error of 34.3 mL. FIG. 6D, subpanels A and B, shows an overview of the LOSO-CV results for the active sensing.

TABLE 2
Overview of the Correlation Between the Temporal
Features and the Fluid in the Joint Space.

| Feature | Pearson's r | Start index | End index |
|---|---|---|---|
| E | 0.55 | 394 | 553 |
| T | 0.74 | 634 | 756 |
| D | 0.71 | 487 | 771 |
| Ae | 0.61 | 333 | 619 |
| St | 0.69 | 561 | 788 |
| S | 0.74 | 561 | 789 |
| Kt | 0.77 | 332 | 785 |
| K | 0.69 | 221 | 589 |

In the study, seven fresh-frozen cadaver specimens with knee replacements were included to investigate the use of active and passive vibration-based techniques to monitor periprosthetic joint effusions. Effusion stages were simulated using saline and bacterial solutions injected into the knee joint space. Active acoustics were recorded during various leg positions, and frequency response functions (FRFs) were calculated between different input/output combinations to assess their sensitivity to the periprosthetic joint fluid. The band power in different frequency bands was extracted as a feature from the FRFs. The most sensitive frequency band for detecting fluid in the joint space was determined to be 1390.5±249.5 Hz resulting from the mid-diaphysis output measurement location and a 45° flexion leg position. A similar frequency range (780-1350 Hz) has been identified as sensitive to structural changes in the knee due to injuries in prior work [39]. This indicates that different structural changes (injuries versus joint fluid) affect the dynamic properties of the tibia differently, as the frequency bands are not identical.

The significant difference in band power between the baseline and 20 mL effusion stage demonstrates that the presented methodology is highly sensitive to extremely small effusions, and potentially much more sensitive than what can be determined during a manual physical examination in the clinic.

The most informative band power was then used in a linear regression model. The model successfully estimated joint fluid volume with a Pearson's r of 0.79, an MAE of 11.1 mL, and a maximum error of 34.3 mL when applying LOSO-CV.

The study was subject to some limitations. First, concerning the normalization of the band power, the preferred approach would have involved utilizing the actual baseline volume of 0 mL for data normalization. However, the accessible data did not have precisely 0 mL and 20 mL volumes for all specimens. Therefore, the next closest fluid volume was used, which was 40 mL, for normalization purposes. While the choice may introduce potential influences on correlation and subsequent modeling outcomes, it was anticipated that any such effects would not be substantial. The expectation arises from the observation that the majority of the data points at the 0 mL baseline exhibit similar normalized band power values. Second, planktonic bacteria were mixed with saline as a test for the presence of bacterial effusion; however, clinically, bacterial effusions (pus) also contain significant bacterial by-products which may alter the mechanical properties of the effusion to a degree that they may be more distinguishable than synovial fluid, than in the cadaveric model. Third, the study's sample size consisted of seven specimens for active sensing and six specimens for passive sensing. To enhance the robustness of the findings and validate the selected sensor locations, leg positioning, frequency bandwidth, and the time segment used for feature extraction. A larger number of specimens may facilitate a more comprehensive comparative analysis between saline and bacteria solutions, potentially unveiling significant differences in the active and passive signals. Fourth, four data points were excluded as they appeared to be noisy or behaved as outliers compared to the remaining data points. It is contemplated that the observation resulted from improper or altered attachment between the transducers and the specimen because low input/output coherence was observed at the lower frequencies. Two of the excluded data points belonged to one specimen that showed little band power sensitivity to the fluid; it was within specimen Pearson's r was 0.01. It is possible that the fluid was not properly injected in the joint space of the specimen, and as a result, did not alter the mechanical properties at the boundary of the tibia, as was the case for the other specimens. When including all data points, the Pearson's r between the band power and the fluid reduced to 0.61. The within-specimen Pearson's r varied between, 0.66 and 1.00, not considering the insensitive specimen discussed earlier. The model's LOSO-CV MAE increased to 14.8 mL with a Pearson's r of 0.61. Lastly, in the cadaveric study, saline was used as a proxy for synovial fluid, the preservation protocols of the cadaveric specimen as well as differences between saline and synovial fluid may alter the sensitivity and specificity of the approach when translated to live subjects; however, the premise and potential of the approach to identify small joint effusions and discriminate between effusion size would remain the same. It is contemplated that conducting validation studies in live subjects will yield valuable insights into the user-friendliness and real-world effectiveness of the proposed approach. The presented active and passive approaches demonstrate the feasibility of developing future wearable effusion monitoring systems to timely inform orthopedic surgeons about the presence and quantification of small periprosthetic effusions.

### Example #B: Non-Invasive Active Acoustics as Biomarkers of Early Periprosthetic Joint Effusions, a Cadaveric Study

The study also evaluated the early periprosthetic joint effusions (Example #B). Details and results are provided in relation to FIGS. 7A-7B. FIG. 7A shows a scatter plot between the normalized BP and the injected fluid volume. The BP correlates with the injected volume with a Pearson's r of 0.83 at a population level (n=33). FIG. 7B illustrates an example of a cadaveric set-up to record joint acoustic emission.

Acoustic emission and electrical bioimpedance techniques show great potential to offer a low-cost, non-invasive, and effective solution to conduct surveillance for PJI by providing objective point of care information on the presence and characteristics of a knee effusion or potentially identify the presence and characteristics of biofilm on the surface of an implant. Such noninvasive methods which could eventually be incorporated into a sleeve or brace, could improve the timely detection of acute PJI, reduce the morbidity of delayed PJI treatment, help confirm PJI eradication, and thus guide patient-specific treatment protocols. In turn, these modalities could be a useful adjunct to PJI diagnosis and potentially guide treatment with less invasive strategies such as serial irrigation and debridement with implant retention or 1-stage revision surgery along with more targeted antibiotic therapy.

### Methods

Seven fresh frozen hip-to-toe cadaver specimens were included in the study. The specimens were obtained from individuals having an average age of 80.6±10.2 years and BMI 26.0±4.9 kg/m² and the specimens were fully thawed overnight prior to the experiments. All seven specimens had knee replacements: three cruciate-retaining total knee arthroplasties (TKA), three posterior stabilizing TKA and one unicondylar partial knee arthroplasty. Different levels and types of effusion stages were simulated by injecting up to 80 ml with 20 ml increments of saline and methicillin-susceptible Staphylococcus aureus (MSSA, 108 CFU/ml) solutions into the joint space. Active acoustics were recorded at each effusion stage, while the specimens were in a supported 45° flexion position. The tibia was excited by a miniature vibration motor (B-81, Radioear, Denmark) that was attached to the skin at the medial surface of the mid-diaphysis. A swept frequency cosine excitation signal with a frequency band between 200 Hz and 5000 Hz and a total duration of 30 seconds was used to excite the tibia. A force transducer (1022V, Dytran, USA) was placed between the vibration motor and the skin to measure the input force of the vibration motor. The output acceleration was measured using a lightweight accelerometer (3225F7, Dytran, USA) approximately 2.5 cm proximal to the input. Both sensors and the motor were attached to the skin using double-sided adhesive tape and an elastic band surrounding the lower leg. FIG. 7B shows an overview of the experimental setup. Input-output frequency response functions (FRF) were calculated and the normalized spectral band power (BP) in varying frequency bands was extracted from the FRFs for each effusion step. Pearson's correlation coefficients (r) were calculated between the BP and the injected volume. A two-sample t-test was used to compare means of normally distributed samples at different injection levels, otherwise, a Wilcoxon rank sum test was used.

A total of 35 effusion stages were measured over all specimens, two data points were excluded due to insufficient measurement quality. The BP in the 1255±228 Hz spectral band showed to be highly correlated (Pearson's r=0.83) with the injected volume over all specimens as shown in FIG. 7A. Within the specimen Pearson's r varied between 0.80 and 1.00. The BP corresponding to 20 ml and 40 ml injected volume showed to be significantly higher than the baseline (0 ml) (p=0.030 and p=0.001, respectively). The BP showed to be on average lower for purely bacteria solution compared to purely saline solution, however, this difference was not significant.

Discussion. The high correlation between BP and injected fluid volumes demonstrates the potential utility of wearable and non-invasive active joint acoustics to quantify extremely small (20 ml) effusions in patients with total knee arthroplasties. The use of active acoustics to discriminate bacteria and saline effusions demonstrates promise but requires more data to be confirmed. The presented approach demonstrates the feasibility of developing future wearable effusion monitoring systems to timely inform orthopedic surgeons on the presence and potentially the characterization of small periprosthetic effusions.

### Example #C: Designing a High Input-Impedance Buffer for Dry-Electrode Bioimpedance Analysis

The study also developed and evaluated a high input-impedance buffer for dry-electrode bioimpedance analysis (Example #C). Details and results are provided in relation to FIGS. 8A-8E. FIGS. 8A-8B illustrate a schematic overview of the AD5940 4-electrode biosensing readout circuit with a simplified electrode-tissue interface for kHz-range applications (FIG. 8A) and a bootstrapped, dual unity-gain buffer configuration using feedback to increase input-impedance at 5 kHz (FIG. 8B). FIG. 8D illustrates measured impedance errors due to Common-Mode to Differential-Mode conversion for several voltage-buffer input stages. FIG. 8E illustrates three trial-averaged, 30-minute real bioimpedance measurements for one subject with interleaved acquisition for the two voltage inputs at 5 kHz. All measurements are acquired at 15-second intervals. The bootstrapped voltage buffer was inserted (or removed) from the system between these samples to interleave and characterize both the buffer and the baseline AD5940 performance while directly controlling for the electrode impedances.

Recent advances in wearable sensing platforms and robust, dry-electrode materials have enabled adhesive-free, continuous bioimpedance sensing. However, long-term continuous monitoring of bioimpedance requires high-impedance dry electrodes, which can exacerbate input-impedance mismatch errors.

The instant study developed a modified mathematical model and design methodology for designing a high input-impedance buffer to improve voltage sensing in four-electrode bioimpedance applications. The bootstrapped buffer design developed in the study confers nearly 75% less common-mode to differential-mode conversion at low frequencies and 50% less error at high frequencies based on the worst-case model. The voltage buffer was directly used and alternated with the baseline inputs of a commercially available off-the-shelf integrated circuit (AD5940) to determine the extent of transient bioimpedance errors emerging from the input-impedance mismatch.

The study evaluated the developed device on two young male adults and showed the system can reduce the time for the 5% convergence time of the final bioimpedance by 100.7 seconds on average. The voltage buffer typically reduces bioimpedance errors by 20% compared to the baseline inputs based on exponential decay curves of best-fit in dry-electrode applications.

Bioimpedance discussion. Bioimpedance analysis has been used in a wide variety of health applications for quantifying the electrical properties of living tissue [1′]. Historically, the most common clinical application is quantifying the body tissue composition of patients [2′]. Other applications include Impedance Cardiography (ICG) for assessing stroke volume [3′], Impedance Pneumography (IPG) for quantifying respiration rate/tidal volume [4′], and joint bioimpedance for assessing knee health [5′]. Bioimpedance-based joint assessment can provide quantitative information about the subject's knee mid-activity per Hersek et al. [5′]. However, such applications typically require gel-electrodes to minimize skin-electrode interface impedance and impedance mismatch.

For longitudinal continuous monitoring of knee impedance, gel-electrodes are unsuitable since they lose their adhesion and irritate the subject's skin [6′]. In a study by Nichols et al., several dry electrode materials have been characterized for continuous bioimpedance sensing [7′], of which silver silicone (i.e., Silitex) electrodes conferred the best performance and yielded a skin-electrode impedance comparable to wet electrodes. Bioimpedance measurement errors largely stem from high skin-electrode impedances, which is common to dry electrodes [8′]. After placement, a significant settling time must be waited before these skin-electrode impedances stabilize within a lower, functional range [9′]. High skin-electrode impedance introduces significant measurement errors, thus requiring waiting for the settling period before consistent measurements can be acquired. In clinical settings, where time is constrained, such a long settling period can be limiting.

High-fidelity bioimpedance is measured using a tetrapolar configuration where two electrodes apply an excitation signal, and the other two measure the potential difference due to that signal. The voltage-sense nodes must have very high input impedance to prevent measurement errors and signal leakage. Since the voltage-sensing circuit confers a finite input impedance due to input capacitance and mega-ohm DC biasing resistors, it directly degrades the desired signal due to Common-Mode to Differential-Mode (CM-DM) Conversion [10′]. While CM-DM conversion is typically cited in the context of ECG and EEG applications for reducing 60 Hz mains coupling, it is especially relevant to bioimpedance designs that bias the system using a single-ended stimulus/source, such as with the AD5940 SoCs. Per [10′] and [11′], maximizing input impedance and CMRR is critical for minimizing CMDM conversion errors.

In the instant study, a high input-impedance buffer is designed and tested with a representative worst-case model circuit to quantify the worst-case performance of such a buffer in the presence of an input-impedance mismatch. The performance difference of such a buffer over the previous baseline system was then presented and characterized by monitoring the performance of both buffers via the transient knee bioimpedance response over 30 minutes for two subjects using dry electrodes.

Modeling Tetrapolar Bioimpedance Measurement Errors. A high-level system overview is described in FIGS. 8A-8B. The negative current electrode, I−, sinks the signal current generated via the excitation amplifier. The excitation amplifier output feeds directly to the current source electrode, I+, and provides the 600 mV AC stimulus voltage. The AD5940 architecture is not truly a differential voltage/current drive. It is single-ended and sinks the signal current via a virtual ground (i.e., V_{bias}). Hence, the current-drive loop is constantly introducing a common-mode voltage to the system as it provides a voltage stimulus.

From FIG. 8A, the voltage common-mode error can be described per Equation 6

$${V}_{e r r o r} = {V}_{c m} \cdot ( \frac{1}{CMRR} + \frac{( {Z}_{V +} - {Z}_{V -} )}{{Z}_{i n}} ) \qquad ( Eq . 4 )$$

To maximize system performance, input-impedance mismatch must be minimized while maximizing both the voltage-buffer input impedance and CMRR. In low-bandwidth (<1 kHz) biosensing applications such as ECG and EMG, it is critical to achieve high input impedance on the order of several GQs [8′], [12′]. This also applies to multi-frequency bioimpedance analysis (MFBIA), where input capacitance typically limits the buffer's input impedance across the frequencies of interest (several kHz to hundreds of kHz). At low frequencies (several kHz), the DC-biasing resistors limit the input-impedance. Feedback can be used for significantly improving the input impedance (i.e., bootstrapping), as demonstrated by Healey et al. [13′]. The bootstrapped unity-gain buffer tested in the instant study is shown and described in FIG. 8B.

The effective impedance error, due to Common-Mode to Differential-Mode conversion, can be expressed as Equation 7.

$${Z}_{e r r o r} = ( {Z}_{I -} + \frac{1}{j ω {C}_{i s o}} ) \cdot ( \frac{1}{CMRR} + \frac{( {Z}_{V +} - {Z}_{V -} )}{{Z}_{i n}} ) \qquad ( Eq . 7 )$$

The most significant implication of the model is the current sink electrode's impedance on the sensed bioimpedance error. If large, the current sink electrode impedance (Z_{I-}) disperses most of the stimulus voltage, increasing the common-mode voltage applied to the buffer while also decreasing the sensed current. This informs the worst-case testing of the voltage buffers, which is similar to the mismatched skin-electrode model testing described by Fortune et al. [12′]. Additionally, for the CM-DM conversion test/model circuit, the tissue impedance is modeled as a simple short. The resulting bioimpedance measured should ideally be zero if both voltage leads have the same impedance. Any differential voltage measured is therefore purely error.

### Methods

Evaluating CM-DM Conversion. To evaluate both systems under the worst-case CM-DM ratio, Z_{I-} was set to a realistic, large impedance while varying both voltage-sense electrode impedances (Z_{V+} and Z_{V-}).

Per [8′][12′][14′], the most significant component of a dry electrode's skin-interface impedance is the capacitive component. This typically can range from 1 nF to several 100 nF depending on the electrode geometry and material properties, as described by Fortune et al [12′]. Per Fortune, the real skin-electrode impedance is commonly modeled with less than a 1 kΩ resistor.

Kaufmann et al. [9′] have characterized dry two-electrode impedances over time, finding it is predominantly capacitive at lower frequencies. The authors of [9′] hypothesized this is the result of the skin-electrode interface wetting over time. While the electrode wets over time, the capacitance increases and resistance decreases, resulting in a net electrode impedance decrease. Based on the results from [9′], the impedance mismatch of the simplified skin-electrode model is modeled via mismatched capacitances and a simplified, two-component model illustrated in FIG. 8A.

The current source electrode is a 1 kΩ resistor in series with 100 nF. The current sink electrode is modeled as a 1 kΩ resistor in series with a 1 nF capacitor to model the worst-case estimate of the negative current electrode reactance according to the models described by Fortune et al. [12′]. Each of the voltage electrodes is comprised of a 1 kΩ resistor in series with a capacitor. To assess positive voltage lead impedance error, the capacitance on the positive voltage lead is decreased from 34 nF to 3.2 nF, including all reactances at 5 kHz from −j2 kΩ to −10 kΩ in −j2 kΩ increments. The negative voltage electrode did not contain any reactance and was modeled as a 1 kΩ resistor. To measure the impact of negative voltage electrode impedance, the mismatch tests were repeated by increasing the reactance on the negative electrode while not adding reactance to the positive electrode. Measurements were acquired at both 5 kHz and 100 kHz.

The above input reactance mismatch testing was repeated for our two test systems utilizing the same AD5940 SoC. As a baseline, impedance measurements were acquired by directly connecting the voltage electrodes to the voltage sense inputs. A low-input capacitance (0.4 pF), high-CMRR, and high-bandwidth unity-gain buffer is presented using the LTC6262 in FIG. 8B. A tradeoff of the opamp is the large 100 nA bias current needed for typical operation, thereby requiring low resistance biasing resistors (3MΩ) and undesirably reducing the effective input impedance at low frequencies. To improve the input impedance at 5 kHz, the buffer output bootstraps the input via feedback to the biasing resistors with a 10 nF capacitor, as described by [13′]. Based on LTspice simulations of the buffer schematic shown in FIG. 16B, the input impedance is increased to around 45 MΩ at 5 kHz.

Human Subject Knee Measurements. Institutional Review Board (IRB) approval was obtained from the Georgia Institute of Technology (IRB #H20329) and all participant populations and protocol methods were enabled in accordance with the approved study protocol. Two adult, healthy, male subjects with no prior history of knee injury were enrolled in the study. Left knee impedance was measured using the same knee-brace tested in [7′]. Dry, 2 cm-by-2 cm Silitex electrodes were used in the knee-brace. The brace was tested on each subject's left knee over three 30-minute trials. The subject's knee bioimpedance was initially monitored until it converged to approximately 100 at 5 kHz, after which the 30-minute measurement period would begin.

To assess the error improvement of using a high-input impedance buffer, a breadboard was used to manually disconnect (or reconnect) the bootstrapped unity-gain buffer at 15-second intervals. Timings are approximated via the SAMD21 microcontroller. Interleaving the system measurements controls for the impedance variation at the dry-electrodes within a single run. The resulting bioimpedances, measured by the voltage buffer vs. the baseline AD5940 inputs, are used to characterize how the errors measured are influenced by the voltage electrode input impedance.

### Results

System Calibration. Both the baseline AD5940 and the bootstrapped unity-gain buffer system were calibrated at each frequency using the 6-parameter method described by Mabrouk et al. [15′].

Worst-Case CM-DM Conversion vs. Inputs. FIGS. 8C and 8D summarize the magnitude of the modeled error when using the baseline AD5940 inputs in addition to the voltage buffer described in FIG. 8B. At 5 kHz (FIG. 8C), the baseline system confers the highest CM-DM conversion error, peaking at near 300Ω. The bootstrapped LTC6262 unity-gain buffer noticeably improves the CM-DM conversion error compared to the baseline system. The impedance error, in this case, ranges from 60-100Ω in the worst case.

At 100 kHz (FIG. 8D), the baseline AD5940 continues to confer the most CM-DM conversion, near 17Ω. This is most likely due to high input capacitance on the order of several picofarads. The bootstrapped LTC6262 buffer confers the lowest CM-DM Conversion at 100 kHz, reaching a maximum of 6.5Ω. Across both 5 kHz and 100 kHz, the bootstrapped buffer confers more than 50% less CM-DM impedance error than the baseline system, as summarized in FIG. 8D. The substantial reduction in measurement error due to electrode-interface impedance mismatch allows for more liberal electrode design, especially in applications where controlling for electrode characteristics across all four electrodes is not possible.

Real-Time Performance of a Bootstrapped Buffer vs. Baseline System. For each subject, all three runs for the bootstrapped voltage buffer and the baseline system were averaged. This is to reduce the risk of overfitting each of the individual runs with an exponential decay curve. The average, real bioimpedance at each point for the averaged runs is summarized in FIG. 8E. For each of the inputs, a least-squares exponential best fit is done. When the bioimpedance transient response converges to the “true” value as time approaches infinity, it decays from a larger, initial impedance. This transient response can be modeled per Equation 8.

$$Z ( t ) = A \cdot exp ( - \frac{t}{τ} ) + {Z}_{∞} \qquad ( Eq . 8 )$$

As a standardized metric, the time for this impedance to converge within 5% of the final value is computed. These parameters are summarized below in Table 3. Table 4 summarizes the time improvement conferred by the unity-gain buffer for the bioimpedance to converge to within 7%, 5%, and 3% of Z for each subject.

TABLE 3
Exponential Fit Parameters for 3-Trial Averaged Runs

| Subject | Parameters | | | | |
|---|---|---|---|---|---|
| Number | Inputs | A[Ω] | τ[s] | Z_{∞} [Ω] | 5% Convergence [s] |
| #1 | UGB | 14.5 | 307.8 | 67.6 | 447.5 |
| | Baseline | 20.0 | 313.3 | 69.1 | 550.7 |
| #2 | UGB | 7.8 | 351.2 | 55.5 | 361.7 |
| | Baseline | 9.7 | 369.7 | 56.0 | 459.9 |

TABLE 4
Bioimpedance Convergence Improvement
Time for Several Error Levels

| Subject | Relative Error Level | | |
|---|---|---|---|
| Number | 7% [s] | 5% [s] | 3% [s] |
| #1 | 101.4 | 103.2 | 106.1 |
| #2 | 91.2 | 98.2 | 107.6 |

As shown in FIG. 8E, the bootstrapped buffer demonstrates a visible reduction in bioimpedance error across both subjects. Per Table 4, the reduction in the 5% convergence time afforded by the unity-gain buffer is 103.2s and 98.2s for subjects 1 and 2, respectively. Between these two subjects, the average improvement of the 5% convergence time is 100.7 seconds when using the bootstrapped unity-gain buffer. From Table 3, the ratio of A[Ω] for each input suggests the unity-gain buffer confers 27.5% and 19.6% less error for subjects 1 and 2, respectively.

In this work, a high input-impedance buffer was present to mitigate common-mode errors present during dry-electrode, MFBIA applications. The buffer design presented confers nearly 75% less common-mode to differential-mode conversion at low frequencies, and 50% less error at high frequencies using the worst-case circuit model.

A high-input impedance buffer was deployed in a small human subject study measuring knee bioimpedance. This buffer was compared against the performance of the baseline AD5940 voltage sensing inputs. For a sample of two young male adults, the system was demonstrated to reduce the time for converging within 5% of the final bioimpedance by 100.7 seconds on average. After modeling the exponential decay of this convergence, the rate of convergence is shown to be a property unique to each subject's skin, such as perspiration rate and hair density [7′].

The exemplary bioimpedance sensor and device, among other features in this study, may be employed in the exemplary system and method described herein.

Discussion Total joint arthroplasty (TJA) of the hip and knee has been shown to consistently relieve pain and improve mobility and quality of life for patients with end-stage degenerative joint disease [1′]-[3′]. Unfortunately, approximately 1-2% of all TJA patients develop a prosthetic joint infection (PJI) following surgery, a devastating complication with severe health and socioeconomic implications. PJI can be challenging to diagnose and typically requires costly, resource-intensive surgical intervention, sometimes more than once, for successful treatment. The current failure rate of PJI treatment is estimated to be roughly 20%, which may, in part, be due to delayed recognition of PJI, and a poor understanding of when a PJI has been eradicated. PJI is the most prominent reason for TKA revision in the first 2 years. PJI is responsible for 15% and 25% of all revision hip and knee procedures, respectively, and is associated with a five-year mortality rate higher than that of breast cancer, melanoma, Hodgkin's lymphoma, and several other common malignancies. In addition to the significant morbidity and mortality associated with PJI, the cost of treating PJI is substantial, with an annual projected cost of treating PJI of $1.85B in the United States alone by 2030 [4′], [5′].

Over the past decade, several groups have aimed to generate a standardized definition of PJI [6′]-[9′]. However, there is currently no single reliable noninvasive test that is both sensitive and specific to diagnosing PJI or for testing whether an infection has been eradicated during treatment of PJI. Currently, the diagnosis and treatment of PJI are assessed using a combination of clinical signs and symptoms, and most commonly with the use of peripheral non-specific biomarkers (erythrocyte sedimentation rate (ESR), c-reactive protein (CRP)), and synovial fluid culture and aspiration results [10′]. One of the limitations of such a diagnostic approach includes obtaining conflicting laboratory values, thereby clouding the certainty of a PJI diagnosis. Additionally, inflammatory markers and synovial fluid aspiration results may be negative in the early stages of clinical infection, in patients with specific low virulence or hard-to-grow organisms, or in patients who have already received antibiotic therapy. Regarding methods for detection of infection eradication, current PJI treatment modalities involve prolonged parenteral antibiotics or placement of antibiotic-impregnated cement spacers, which in turn may alter traditional proinflammatory threshold values and other traditional metrics used to diagnose PJI. Most importantly, all the currently available methods are either subjective tests that rely on the caregiver's experience or objective laboratory tests that are nonspecific or require lead time which can be detrimental for a time-sensitive pathogen.

Vibration analysis and acoustic emission (AE) analysis are nondestructive testing techniques that are widely used and recognized in engineering to evaluate the structural integrity of a broad spectrum of mechanical structures. The process of implementing a structural integrity monitoring strategy involving periodically spaced dynamic response analysis is referred to as Structural Health Monitoring (SHM) in the existing literature [11′].

Vibration, or modal, analysis is a non-destructive technique that investigates the structural behavior of mechanical systems by measuring the dynamic response and extracting modal parameters, i.e., natural frequencies, mode shapes, and damping ratios [12′], [13′]. This dynamic response is usually provided by stimulating the structure of interest using an external vibration source. AE analysis is a specific wave-based method to analyze transient elastic waves generated within a material due to the rapid release of energy from local sources (e.g., cracks or friction), and is used to assess the structural properties often at ultrasonic frequencies (>20 kHz) in solid mechanical structures [14′].

In musculoskeletal sensing applications, vibration-based techniques offer a non-invasive and convenient technology to assess the structural health of the musculoskeletal system; more specifically, these techniques could be used to identify intraarticular pathology. Transducers placed on the surface of the skin can be used to capture the dynamic response of bones and joints. Such techniques have successfully been studied in the past for a variety of musculoskeletal conditions. Existing studies have demonstrated the usability of vibration analysis to monitor bone fracture healing [15′], [16′] or to assess the effect of meniscus tears and meniscectomy on the dynamic response [17′]. Other studies have demonstrated the use of vibration-based technologies to assess and monitor the stability of dental implants [18′], [19′], and hip and knee implants [20′]-[26′]. Previous work has demonstrated that vibration-based technologies utilizing an excitation source are able to quantify the dynamic characteristics of the knee joint in a repeatable manner [27′] and to accurately estimate Achilles tendon loading [28′].

AE-based techniques have also been investigated in the realm of musculoskeletal sensing applications. AEs, often referred to interchangeably as joint acoustic emissions (JAEs), facilitate analyzing the vibrations associated with joint articulation. These acoustic signals are related to surface friction [29′] and can be used to extract an index of joint structure and health using machine learning algorithms. Recent ex vivo and in vivo studies have revealed that, by studying characteristics of the acoustic emissions of the knee, meniscus tears can be detected [30′], [31′], and that knee involvement in juvenile idiopathic arthritis [32′]-[34′] and rheumatoid arthritis [35′] can be assessed. When implementing these vibration-based techniques in wearable form factors, a non-invasive diagnostic method can be deployed at the point of care for ubiquitous use, such as at home [12′].

Regarding applications in total knee arthroplasty (TKA), vibration analysis approaches have been used to characterize squeaking, identify material wear of polyethylene, gross implant loosening, and potentially time since implantation [36′], [37′]. Currently, the utility of vibration-based techniques to identify PJJ in TKA remains unexplored [25′].

Electrical bioimpedance (EBI) is the measure of the electrical conductivity of the tissue and has been used in the literature to quantify the changes in soft tissue characteristics and fluid composition. EBI has been used extensively in studying static changes in body mass composition analysis due to its ability to differentiate between fluid, fat, and muscle content of the tissue. The Inan Research Lab has devised a novel EBI method by measuring the dynamic changes in EBI during knee range of motion and has validated the potential for EBI to detect changes in the contents of a knee effusion [17′], [18′]. Regarding applications in total knee arthroplasty, to date, AE and EBI approaches have been used to identify material wear of polyethylene, gross implant loosening, and potentially time since implantation [19′], [20′]. Currently, the utility of using AE and EBI to identify PJI in Total Knee Arthroplasty (TKA) remains unexplored [21′].

The exemplary system and method can provide a reliable, non-invasive, inexpensive, objective, quick, and convenient assessment of joint pathologies and/or joint conditions as compared to the existing techniques to assess/monitor joint pathologies, such as laboratory tests, subjective manual palpation, ultrasound that requires training and expensive equipment.

The exemplary system and method can employ a fusion of active acoustic and bioimpedance operation, while existing work has focused on passive acoustics (in combination with EBI). Active acoustic differs from passive acoustics in the utilization of an input source to actively excite the structure of interest. In contrast, passive acoustics focuses on the intrinsic vibrations produced by the joint, such as its natural movements. These movements are inherently dependent on the patient, leading to uncontrolled variations between different individuals. Active acoustics have advantages over passive acoustics since they enable to repeatedly excite the structure with a controlled and sufficient amount of energy (high signal-to-noise ratio) that does not rely on any movements of the patient. The controlled excitation of AA also permits the concentration on specific dynamic ranges. This comprehensive approach facilitates more sensitive detection of minor structural alterations in the joint compared to the currently available techniques.

U.S. Patent Publication No. US20180160966A1 discloses the use of passive joint sounds and bioimpedance to assess joint health.

Noted in Bolus [28′], active vibration sensing was used as a technique to monitor Achilles Tendon loading/tension using soft tissue tension assessment via single frequency analysis and does not use bioimpedance.

U.S. Pat. No. 10,709,377B2 discloses a system to record sensor data from the knee using a wearable patch was described to share post-op, at-home activity data with a clinical team.

In other publications, a wearable device is disclosed that allows personal monitoring of the rehabilitation after knee replacement surgery that collects a range of motion data, step count, and pain scores and provides the data to a surgeon and care team; however, active vibration sensing nor bioimpedance data are included.

In a publication by Pichonnaz [17′], the use of bioimpedance spectroscopy to monitor effusions post-TKA was evaluated using a benchtop system to compare bioimpedance between the affected leg and healthy leg. There was no disclosure for a fuse vibration analysis with bioimpedance analysis, which is dissimilar from the exemplary method and may not have to be derived from a contralateral comparison and may use a relative metric calculated from the bioimpedance data of a single leg. The exemplary method may also consider effusions within the joint space to differentiate it from overall tissue swelling (edema) post-surgery.

In Yau [18′], the current non-invasive techniques to assess swelling/effusions after TKA are summarized, including (1) volumetric measurements using manual circumferential measurements, (2) MRI imaging, (3) bioimpedance spectroscopy, (4) optometric measurements, (5) ultrasound imaging. There may be performance and or practical limitations: manual volumetric measurements can suffer from operator bias and do not differentiate well between tissue edema and joint effusion; MRI imaging is expensive and time-consuming for routine assessment; bioimpedance spectroscopy needs further development and data augmentation to result into sufficient diagnostic performance and differentiation between, tissue edema and joint space effusions; optometric measurements provide overall 3D image of joint, basically an objective volumetric measurement, but do not differentiate between tissue edema and joint effusions; ultrasound imaging requires specific equipment and trained personnel that depends on the operator's experience.

Whereas, the exemplary system and method can target joint space effusions and specifically, offers quick and objective effusion measures based on vibration and bioimpedance-based biomarkers, and does not require bulky and expensive equipment.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Example Computing System. An example computing device upon which the analysis system described herein may be implemented is illustrated in FIG. 1. It should be understood that the example computing device is only one example of a suitable computing environment upon which the methods described herein may be implemented. Optionally, the computing device can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, a computing device typically includes at least one processing unit and system memory. Depending on the exact configuration and type of computing device, system memory may be volatile (such as random access memory (RAM)), non-volatile (such as read-only memory (ROM), flash memory, etc.), or some combination of the two. The processing unit may be a programmable processor that performs arithmetic and logic operations necessary for the operation of the computing device. The computing device may also include a bus or other communication mechanism for communicating information among various components of the computing device.

Computing device may have additional features/functionality. For example, computing device may include additional storage such as removable storage and non-removable storage, including, but not limited to, magnetic or optical disks or tapes. Computing device may also contain network connection(s) that allow the device to communicate with other devices. Computing device may also have input device(s) such as a keyboard, mouse, touch screen, etc. Output device(s), such as a display, speakers, printer, etc., may also be included. The additional devices may be connected to the bus in order to facilitate the communication of data among the components of the computing device. All these devices are well-known in the art and need not be discussed at length here.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Examples of tangible, computer-readable media may include, but is not limited to, volatile media, non-volatile media, removable media, and non-removable media implemented in any method or technology for storage of information such as computer-readable instructions, data structures, program modules or other data. System memory, removable storage, and non-removable storage are all examples of tangible, computer storage media. Examples of tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 406 may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising,” “containing,” or “including,” is meant that at least the name compound, element, particle, or method step is present in the composition or article or method but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications as listed below and throughout this document are hereby incorporated by reference in their entirety herein.

### Reference List #1

[1]D. B. Bumpass and R. M. Nunley, “Assessing the value of a total joint replacement,” Curr Rev Musculoskelet Med, vol. 5, no. 4, Art. no. 4, December 2012, doi: 10.1007/s12178-012-9139-6.
[2]A. J. Carr et al., “Knee replacement,” The Lancet, vol. 379, no. 9823, Art. no. 9823, April 2012, doi: 10.1016/S0140-6736(11)60752-6.
[3]I. D. Learmonth, C. Young, and C. Rorabeck, “The operation of the century: total hip replacement,” vol. 370, p. 12, 2007.
[4]M. Rezapoor and J. Parvizi, “Prevention of Periprosthetic Joint Infection,” The Journal of Arthroplasty, vol. 30, no. 6, Art. no. 6, June 2015, doi: 10.1016/j.arth.2015.02.044.
[5]M. Sloan, A. Premkumar, and N. P. Sheth, “Projected Volume of Primary Total Joint Arthroplasty in the U.S., 2014 to 2030,” The Journal of Bone and Joint Surgery, vol. 100, no. 17, Art. no. 17, September 2018, doi: 10.2106/JBJS.17.01617.
[6]C. D. Valle et al., “Diagnosis of Periprosthetic Joint Infections of the Hip and Knee:” American Academy of Orthopaedic Surgeon, vol. 18, no. 12, Art. no. 12, December 2010, doi: 10.5435/00124635-201012000-00006.
[7]J. Parvizi et al., “New Definition for Periprosthetic Joint Infection: From the Workgroup of the Musculoskeletal Infection Society,” Clinical Orthopaedics & Related Research, vol. 469, no. 11, Art. no. 11, November 2011, doi: 10.1007/s11999-011-2102-9.
[8]D. R. Osmon et al., “Executive Summary: Diagnosis and Management of Prosthetic Joint Infection: Clinical Practice Guidelines by the Infectious Diseases Society of Americaa,” Clinical Infectious Diseases, vol. 56, no. 1, Art. no. 1, January 2013, doi: 10.1093/cid/cis966.
[9]J. Parvizi et al., “The 2018 Definition of Periprosthetic Hip and Knee Infection: An Evidence-Based and Validated Criteria,” The Journal of Arthroplasty, vol. 33, no. 5, Art. no. 5, May 2018, doi: 10.1016/j.arth.2018.02.078.
[10]J. Marschall, M. A. Lane, S. E. Beekmann, P. M. Polgreen, and H. M. Babcock, “Current management of prosthetic joint infections in adults: results of an Emerging Infections Network survey,” International Journal of Antimicrobial Agents, vol. 41, no. 3, Art. no. 3, March 2013, doi: 10.1016/j.ijantimicag.2012.10.023.
[11]C. R. Farrar and K. Worden, Structural health monitoring: a machine learning perspective. Chichester, West Sussex, U.K.; Hoboken, N.J: Wiley, 2013.
[12]P. Avitabile, Modal Testing: A Practitioner's Guide: A Practitioner's Guide. Chichester, UK: John Wiley & Sons Ltd, 2017. doi: 10.1002/9781119222989.
[13]W. Heylen and P. Sas, Modal Analysis Theory and Testing. Katholieke Universiteit Leuven, Departement Werktuigkunde; Leuven, 2006.
[14]C. J. Hellier, Handbook of nondestructive evaluation, Third edition. New York: McGraw-Hill, 2020.
[15]L. Mattei, F. Di Puccio, and S. Marchetti, “Fracture Healing Monitoring by Impact Tests: Single Case Study of a Fractured Tibia With External Fixator,” IEEE Journal of Translational Engineering in Health and Medicine, vol. 7, pp. 1-6, 2019, doi: 10.1109/JTEHM.2019.2901455.
[16]L. Nokes, W. J. Mintowt-Czyz, J. A. Fairclough, I. Mackie, and J. Williams, “Vibration analysis in the assessment of conservatively managed tibial fractures,” Journal of Biomedical Engineering, vol. 7, no. 1, Art. no. 1, January 1985, doi: 10.1016/0141-5425(85)90007-X.
[17]M. Safaei, N. B. Bolus, D. C. Whittingslow, H.-K. Jeong, A. Erturk, and O. T. Inan, “Vibration Stimulation as a Non-Invasive Approach to Monitor the Severity of Meniscus Tears,” IEEE Trans. Neural Syst. Rehabil. Eng., vol. 29, pp. 350-359, 2021, doi: 10.1109/TNSRE.2021.3050439.
[18]N. Meredith, K. Book, B. Friberg, T. Jemt, and L. Sennerby, “Resonance frequency measurements of implant stability in vivo,” Clinical Oral Implants Research, no. 8, Art. no. 8, 1997.
[19]L. Sennerby and N. Meredith, “Implant stability measurements using resonance frequency analysis: biological and biomechanical aspects and clinical implications,” Periodontology 2000, vol. 47, no. 1, Art. no. 1, June 2008, doi: 10.1111/j.1600-0757.2008.00267.x.
[20]Q. Goossens et al., “Acoustic analysis to monitor implant seating and early detect fractures in cementless THA: An in vivo study,” J Orthop Res, p. jor.24837, September 2020, doi: 10.1002/jor.24837.
[21]S. Leuridan et al., “Vibration-based fixation assessment of tibial knee implants: A combined in vitro and in silico feasibility study,” Medical Engineering & Physics, vol. 49, pp. 109-120, November 2017, doi: 10.1016/j.medengphy.2017.08.007.
[22]S. Leuridan et al., “Development of an Instrument to Assess the Stability of Cementless Femoral Implants Using Vibration Analysis During Total Hip Arthroplasty,” IEEE J. Transl. Eng. Health Med., vol. 9, pp. 1-10, 2021, doi: 10.1109/JTEHM.2021.3128276.
[23]Q. Goossens et al., “Development of an acoustic measurement protocol to monitor acetabular implant fixation in cementless total hip Arthroplasty: A preliminary study,” Medical Engineering & Physics, vol. 49, pp. 28-38, November 2017, doi: 10.1016/j.medengphy.2017.07.006.
[24]A. Arami, J.-R. Delaloye, H. Rouhani, B. M. Jolles, and K. Aminian, “Knee Implant Loosening Detection: A Vibration Analysis Investigation,” Annals of Biomedical Engineering, vol. 46, no. 1, Art. no. 1, January 2018, doi: 10.1007/s10439-017-1941-2.
[25]L. Khokhlova, D.-S. Komaris, S. Tedesco, and B. O'Flynn, “Assessment of Hip and Knee Joints and Implants Using Acoustic Emission Monitoring: A Scoping Review,” IEEE Sensors J., vol. 21, no. 13, Art. no. 13, July 2021, doi: 10.1109/JSEN.2020.3045203.
[26]L. C. Pastrav, S. V. N. Jaecques, I. Jonkers, G. Van der Perre, and M. Mulier, “In vivo evaluation of a vibration analysis technique for the per-operative monitoring of the fixation of hip prostheses,” Journal of Orthopaedic Surgery and Research, vol. 4, no. 1, Art. no. 1, 2009, doi: 10.1186/1749-799X-4-10.
[27]M. Safaei, N. B. Bolus, A. Erturk, and O. T. Inan, “Vibration Characterization of the Human Knee Joint in Audible Frequencies,” Sensors, vol. 20, no. 15, Art. no. 15, July 2020, doi: 10.3390/s20154138.
[28]N. B. Bolus, H. K. Jeong, B. M. Blaho, M. Safaei, A. J. Young, and O. T. Inan, “Fit to Burst: Toward Noninvasive Estimation of Achilles Tendon Load Using Burst Vibrations,” IEEE Trans. Biomed. Eng., vol. 68, no. 2, pp. 470-481, February 2021, doi: 10.1109/TBME.2020.3005353.
[29]S. Gharehbaghi, H. K. Jeong, M. Safaei, and O. T. Inan, “A Feasibility Study on Tribological Origins of Knee Acoustic Emissions,” IEEE Trans. Biomed. Eng., vol. 69, no. 5, pp. 1685-1695, May 2022, doi: 10.1109/TBME.2021.3127030.
[30]G. C. Ozmen et al., “Detection of Meniscal Tear Effects on Tibial Vibration Using Passive Knee Sound Measurements,” IEEE Trans. Biomed. Eng., vol. 68, no. 7, Art. no. 7, July 2021, doi: 10.1109/TBME.2020.3048930.
[31] Daniel. C. Whittingslow, H.-K. Jeong, V. G. Ganti, N. J. Kirkpatrick, G. F. Kogler, and O. T. Inan, “Acoustic Emissions as a Non-invasive Biomarker of the Structural Health of the Knee,” Ann Biomed Eng, vol. 48, no. 1, Art. no. 1, January 2020, doi: 10.1007/s10439-019-02333-x.
[32]D. C. Whittingslow et al., “Knee Acoustic Emissions as a Digital Biomarker of Disease Status in Juvenile Idiopathic Arthritis,” Front. Digit. Health, vol. 2, p. 571839, November 2020, doi: 10.3389/fdgth.2020.571839.
[33]S. Gharehbaghi, D. Whittingslow, L. A. Ponder, S. Prahalad, and O. Inan, “Acoustic Emissions from Loaded and Unloaded Knees to Assess Joint Health in Patients with Juvenile Idiopathic Arthritis,” IEEE J. Biomed. Health Inform., pp. 1-1, 2021, doi: 10.1109/JBHI.2021.3081429.
[34]Q. Goossens et al., “Knee acoustic emissions as a noninvasive biomarker of articular health in patients with juvenile idiopathic arthritis: a clinical validation in an extended study population,” Pediatr Rheumatol, vol. 21, no. 1, p. 59, June 2023, doi: 10.1186/s12969-023-00842-7.
[35]K. L. Richardson et al., “Quantifying Rheumatoid Arthritis Disease Activity Using a Multimodal Sensing Knee Brace,” IEEE Trans. Biomed. Eng., vol. 69, no. 12, pp. 3772-3783, December 2022, doi: 10.1109/TBME.2022.3177074.
[36]R. Schwarzkopf, F. Kummer, and W. Jaffe, “Acoustic Emission Studies of Posterior Stabilized and Cruciate Retaining Knee Arthroplasties,” J Knee Surg, vol. 24, no. 03, Art. no. 03, September 2011, doi: 10.1055/s-0031-1284730.
[37]G. W. Rodgers et al., “Acoustic Emission Monitoring of Total Hip Arthroplasty Implants,” IFAC Proceedings Volumes, vol. 47, no. 3, Art. no. 3, 2014, doi: 10.3182/20140824-6-ZA-1003.00928.
[38]G. C. Ozmen, M. Safaei, L. Lan, and O. T. Inan, “A Novel Accelerometer Mounting Method for Sensing Performance Improvement in Acoustic Measurements From the Knee,” Journal of Vibration and Acoustics, vol. 143, no. 3, Art. no. 3, June 2021, doi: 10.1115/1.4048554.
[39]G. C. Ozmen et al., “Wearable Active Vibration Sensing for Mid-Activity Knee Health Assessment,” presented at the IEEE 19th International Conference on Body Sensor Networks (BSN), 2023.
[40]P. Welch, “The use of fast Fourier transform for the estimation of power spectra: A method based on time averaging over short, modified periodograms,” IEEE Trans. Audio Electroacoust., vol. 15, no. 2, Art. no. 2, June 1967, doi: 10.1109/TAU.1967.1161901.
[41]S. Leuridan et al., “Determination of replicate composite bone material properties using modal analysis,” Journal of the Mechanical Behavior of Biomedical Materials, vol. 66, pp. 12-18, February 2017, doi: 10.1016/j.jmbbm.2016.10.018.

### Reference List #2

[1′]D. Naranjo-Hernandez, J. Reina-Tosina, and M. Min, “Fundamentals, recent advances, and future challenges in bioimpedance devices for healthcare applications,” Journal of Sensors, vol. 2019, 2019.
[2′]C. H. Ling, A. J. de Craen, P. E. Slagboom, D. A. Gunn, M. P. Stokke!, R. G. Westendorp, and A. B. Maier, “Accuracy of direct segmental multifrequency bioimpedance analysis in the assessment of total body and segmental body composition in middle-aged adult population,” Clinical Nutrition, vol. 30, no. 5, pp. 610-615, 2011.
[3′]A. Sherwood, M. T. Allen, J. Fahrenberg, R. M. Kelsey, W. R. Lovallo, and L. J. Van Doornen, “Methodological guidelines for impedance cardiography,” Psychophysiology, vol. 27, no. I, pp. 1-23, 1990.
[4′]J. A. Berkebile, S. A. Mabrouk, V. G. Ganti, A. V. Srivatsa, J. A. Sanchez-Perez, and 0. T. Inan, “Towards estimation of tidal volume and respiratory timings via wearable-patch-based impedance pneumography in ambulatory settings,” IEEE Transactions on Biomedical Engineering, vol. 69, no. 6, pp. 1909-19 19, 2021.
[5′]S. Hersek, H. Tireyin, C. N. Teague, M. L. Millard-Stafford, H.-K. Jeong, M. M. Bavare, P. Wolkoff, M. N. Sawka, and 0. T. Inan, “Wearable vector electrical bioimpedance system to assess knee joint health,” IEEE Transactions on Biomedical Engineering, vol. 64, no. 10, pp. 2353-2360, 2016.
[6′]R. Kusche, S. Kaufmann, and M. Ryschka, “Dry electrodes for bioimpedance measurements-design, characterization and comparison,” Biomedical Physics & Engineering Express, vol. 5, no. I, p. 015001, 2018.
[7′]C. J. Nichols, S. A. Mabrouk, G. C. Ozmen, A. H. Gazi, and 0. T. Inan, “Validating adhesive-free bioimpedance of the leg in mid-activity and uncontrolled settings,” IEEE Transactions on Biomedical Engineering, 2023.
[8′]Y. M. Chi, T.-P. Jung, and G. Cauwenberghs, “Dry-contact and noncontact biopotential electrodes: Methodological review,” IEEE Reviews in Biomedical Engineering, vol. 3, pp. 106-119, 2010.
[9′]S. Kaufmann, G. Ardelt, and M. Ryschka, “Measurements of electrode skin impedances using carbon rubber electrodes-first results,” in Journal of Physics: Conference Series, vol. 434, p. 012020, IOP Publishing, 2013.
[10′]A. D. Legatt, “Impairment of common mode rejection by mismatched electrode impedances: Quantitative analysis,” American Journal of EEG Technology, vol. 35, no. 4, pp. 296-302, 1995.
[11′]J. Rosell, J. Colominas, P. Riu, R. Pallas-Areny, and J. Webster, “Skin impedance from I hz to I mhz,” IEEE Transactions on Biomedical Engineering, vol. 35, no. 8, pp. 649-651, 1988.
[12′]B. C. Fortune, C. G. Pretty, C. J. Cameron, L. R. McKenzie, L. T. Chatfield, and M. P. Hayes, “Electrode-skin impedance imbalance measured in the frequency domain,” Biomedical Signal Processing and Control, vol. 63, p. I 02202, 2021.
[13′]T. J. Healey, E. R. Billinge, J. J. Alix, V. Kadirkamanathan, and C. R. Kemp, “Circuit architecture for electrical impedance spectroscopy instrumentation to address electrode impedance mismatch in clinical devices,” IEEE Sensors Journal, vol. 21, no. 20, pp. 22258-22269, 2021.
[14′]W. Franks, I. Schenker, P. Schmutz, and A. Hierlemann, “Impedance characterization and modeling of electrodes for biomedical applications,” IEEE Transactions on Biomedical Engineering, vol. 52, pp. 1295-1 302, July 2005.
[15′]S. Mabrouk, S. Hersek, H. K. Jeong, D. Whittingslow, V. G. Ganti, P. Wolkoff, and 0. T. Inan, “Robust longitudinal ankle edema assessment using wearable bioimpedance spectroscopy,” IEEE Transactions on Biomedical Engineering, vol. 67, no. 4, pp. 1019-1029, 2019.
[16′]A. Montalibet and E. McAdams, “A practical method to reduce electrode mismatch artefacts during 4-electrode bioimpedance spectroscopy measurements,” in 2018 40th Annual International Conference of the IEEE Engineering in Medicine and Biology Society (EMBC), pp. 5775-5779, IEEE, 2018.
[17′] Pichonnaz, Claude, et al. “Bioimpedance spectroscopy for swelling evaluation following total knee arthroplasty: a validation study.” BMC Musculoskeletal Disorders 16 (2015): 1-8.
[18′] Yau, L I Ka, et al. “Swelling assessment after total knee arthroplasty.” Journal of Orthopaedic Surgery 30.3 (2022): 10225536221127668.

What is claimed is:

1. A system comprising:
an analysis system comprising:
a processor; and
a memory having instructions stored thereon, wherein execution of the instructions by the processor causes the processor to:
receive a measurement data acquired from a device configured to interrogate energy into a knee and proximate tissue of a joint arthroplasty patient;
determine, by a trained AI model or statistical model, an estimated effusion state of the knee and proximate tissue; and
output, via a report or graphical user interface, the estimated effusion state, wherein the effusion state is employed by a clinician to direct treatment or to diagnosis for an infection of the knee and proximate tissue.

2. The system of claim 1, wherein the measurement data is acquired from impulse or vibratory mechanical energy applied to the knee and/or proximate tissue.

3. The system of claim 1, wherein the measurement data is acquired from an impulse or electrical energy applied to the knee and/or proximate tissue.

4. The system of claim 1, wherein the measurement data is acquired from a shaker, a bioimpedance sensor, or a combination thereof.

5. The system of claim 1, wherein the trained AI model or statistical model comprises a linear regression model.

6. The system of claim 1, wherein the device is configured to induce swept-frequency cosine excitation mechanical input to the knee or proximate tissue, wherein the mechanical input has a primary frequency component between 200 Hz and 5000 Hz and induced for at least 20 seconds.

7. The system of claim 1, wherein the device was positioned at an anteromedial position on the knee, an anterior position of the knee, or at a tibial crest of the knee.

8. The system of claim 1, wherein the analysis system is configured as cloud infrastructure.

9. The system of claim 1, wherein the analysis system is an edge device configured to operate with the device.

10. A method of non-evasively evaluating an effusion state of a knee or proximate tissue comprising;
providing a measurement device positioned at an anteromedial position on the knee, an anterior position of the knee, or at a tibial crest of the knee, wherein the measurement device is configured to (i) direct an impulse or vibratory mechanical energy to the knee and/or proximate tissue and measure resulting impulse or vibratory mechanical energy, (ii) direct an electrical stimulus to the knee and/or proximate tissue and measure resulting electrical measurement for a measure of bioimpedance of the knee and/or proximate tissue, or (iii) a combination thereof;
transmitting measurement data acquired from the measurement device to an analysis system configured to determine, by a trained AI model or statistical model, an estimated effusion state of the knee and proximate tissue;
determining, by the trained AI model or statistical model, an estimated effusion state of the knee and proximate tissue; and
outputting by a report or graphical user interface the estimated effusion state, wherein the effusion state is employed by a clinician to direct treatment or to diagnosis for an infection of the knee and proximate tissue.

11. The method of claim 10 further comprising:
directing aspiration of the knee or proximate tissue when an estimated effusion volume is higher than a normal baseline effusion volume at a post-operation assessment of the knee or proximate tissue.

12. The method of claim 10, wherein the measurement device is positioned when the knee is bent between 300-60°.

13. The method of claim 10, wherein the measurement data is acquired from impulse or vibratory mechanical energy applied to the knee and/or proximate tissue.

14. The method of claim 10, wherein the measurement data is acquired from an impulse or electrical energy applied to the knee and/or proximate tissue.

15. The method of claim 10, wherein the measurement data is acquired from a shaker, a bioimpedance sensor, or a combination thereof.

16. The method of claim 10, wherein the trained AI model or statistical model comprises a linear regression model.

17. The method of claim 10, wherein the measurement device is configured to induce swept-frequency cosine excitation mechanical input to the knee or proximate tissue, wherein the mechanical input had a primary frequency component between 200 Hz and 5000 Hz and was inducted for at least 20 seconds.

18. The method of claim 10, wherein the analysis system is configured as cloud infrastructure or an edge device configured to operate with the measurement device.

19. A system comprising:
an analysis system comprising:
a processor; and
a memory having instructions stored thereon, wherein execution of the instructions by the processor causes the processor to:
receive a measurement data acquired from a device configured to interrogate energy into a knee and proximate tissue of a joint arthroplasty patient;
determine, by a trained AI model, an estimated joint condition of the knee and proximate tissue, wherein the estimated joint condition is selected from the group consisting of periprosthetic joint infections, implant integrity, implant loosening, and disease; and
output by a report or graphical user interface the estimated joint condition, wherein the estimated joint condition is employed by a clinician to direct treatment or to diagnosis of the knee and proximate tissue.

20. The system of claim 19, wherein the device is configured with an accelerometer, a force sensor, a thermal sensor, or combinations thereof.
